SEQUENCE LISTING (56 sequences)

SEQ ID NO: 1 - protein, 21 aa, Artificial Sequence
  Description of Artificial Sequence Synthetic peptide
  MOD_RES (17)..(17): D-amino acid
  MOD_RES (18)..(18): D-amino acid
  MOD_RES (19)..(19): D-amino acid
  MOD_RES (20)..(20): D-amino acid
AINGITNKVNSVIESSPPPPC

SEQ ID NO: 2 - protein, 21 aa, Artificial Sequence
  Description of Artificial Sequence Synthetic peptide
  MOD_RES (2)..(2): D-amino acid
  MOD_RES (3)..(3): D-amino acid
  MOD_RES (4)..(4): D-amino acid
  MOD_RES (5)..(5): D-amino acid
CPPPPSSAINGITNKVNSVIE

SEQ ID NO: 3 - protein, 21 aa, Artificial Sequence
  Description of Artificial Sequence Synthetic peptide
  MOD_RES (17)..(17): D-amino acid
  MOD_RES (18)..(18): D-amino acid
  MOD_RES (19)..(19): D-amino acid
  MOD_RES (20)..(20): D-amino acid
RGFFGAIGFIEGGWTSPPPPC

SEQ ID NO: 4 - protein, 21 aa, Artificial Sequence
  Description of Artificial Sequence Synthetic peptide
  MOD_RES (2)..(2): D-amino acid
  MOD_RES (3)..(3): D-amino acid
  MOD_RES (4)..(4): D-amino acid
  MOD_RES (5)..(5): D-amino acid
CPPPPSTRGFFGAIGFIEGGW

SEQ ID NO: 5 - protein, 23 aa, Artificial Sequence
  Description of Artificial Sequence Synthetic peptide
  MOD_RES (2)..(2): D-amino acid
  MOD_RES (3)..(3): D-amino acid
  MOD_RES (4)..(4): D-amino acid
  MOD_RES (5)..(5): D-amino acid
CPPPPSTNIHPITIGKSPKYVKS

SEQ ID NO: 6 - protein, 23 aa, Artificial Sequence
  Description of Artificial Sequence Synthetic peptide
  MOD_RES (19)..(19): D-amino acid
  MOD_RES (20)..(20): D-amino acid
  MOD_RES (21)..(21): D-amino acid
  MOD_RES (22)..(22): D-amino acid
NIHPITIGKSPKYVKSTSPPPPC

SEQ ID NO: 7 - protein, 16 aa, Artificial Sequence
  Description of Artificial Sequence Synthetic peptide
  MOD_RES (2)..(2): D-amino acid
  MOD_RES (3)..(3): D-amino acid
  MOD_RES (4)..(4): D-amino acid
  MOD_RES (5)..(5): D-amino acid
  MOD_RES (8)..(8): L-Norleucine
CPPPPSSLATGLRNRP

SEQ ID NO: 8 - protein, 16 aa, Artificial Sequence
  Description of Artificial Sequence Synthetic peptide
  MOD_RES (1)..(1): L-Norleucine
  MOD_RES (12)..(12): D-amino acid
  MOD_RES (13)..(13): D-amino acid
  MOD_RES (14)..(14): D-amino acid
  MOD_RES (15)..(15): D-amino acid
LATGLRNRPSSPPPPC

SEQ ID NO: 9 - protein, 19 aa, Artificial Sequence
  Description of Artificial Sequence Synthetic peptide
  MOD_RES (2)..(2): D-amino acid
  MOD_RES (3)..(3): D-amino acid
  MOD_RES (4)..(4): D-amino acid
  MOD_RES (5)..(5): D-amino acid
CPPPPSSRIENLNKKVDDG

SEQ ID NO: 10 - protein, 19 aa, Artificial Sequence
  Description of Artificial Sequence Synthetic peptide
  MOD_RES (15)..(15): D-amino acid
  MOD_RES (16)..(16): D-amino acid
  MOD_RES (17)..(17): D-amino acid
  MOD_RES (18)..(18): D-amino acid
RIENLNKKVDDGSSPPPPC

SEQ ID NO: 11 - protein, 18 aa, Artificial Sequence
  Description of Artificial Sequence Synthetic peptide
  MOD_RES (2)..(2): D-amino acid
  MOD_RES (3)..(3): D-amino acid
  MOD_RES (4)..(4): D-amino acid
  MOD_RES (5)..(5): D-amino acid
CPPPPSSDIWTYNAELLV

SEQ ID NO: 12 - protein, 18 aa, Artificial Sequence
  Description of Artificial Sequence Synthetic peptide
  MOD_RES (14)..(14): D-amino acid
  MOD_RES (15)..(15): D-amino acid
  MOD_RES (16)..(16): D-amino acid
  MOD_RES (17)..(17): D-amino acid
DIWTYNAELLVSSPPPPC

SEQ ID NO: 13 - protein, 19 aa, Artificial Sequence
  Description of Artificial Sequence Synthetic peptide
  MOD_RES (2)..(2): D-amino acid
  MOD_RES (3)..(3): D-amino acid
  MOD_RES (4)..(4): D-amino acid
  MOD_RES (5)..(5): D-amino acid
CPPPPSSEIGNGSFEFYHK

SEQ ID NO: 14 - protein, 19 aa, Artificial Sequence
  Description of Artificial Sequence Synthetic peptide
  MOD_RES (15)..(15): D-amino acid
  MOD_RES (16)..(16): D-amino acid
  MOD_RES (17)..(17): D-amino acid
  MOD_RES (18)..(18): D-amino acid
EIGNGSFEFYHKSSPPPPC

SEQ ID NO: 15 - protein, 16 aa, Artificial Sequence
  Description of Artificial Sequence Synthetic peptide
  MOD_RES (2)..(2): D-amino acid
  MOD_RES (3)..(3): D-amino acid
  MOD_RES (4)..(4): D-amino acid
  MOD_RES (5)..(5): D-amino acid
CPPPPTSIGYHANNST

SEQ ID NO: 16 - protein, 14 aa, Artificial Sequence
  Description of Artificial Sequence Synthetic peptide
  MOD_RES (10)..(10): D-amino acid
  MOD_RES (11)..(11): D-amino acid
  MOD_RES (12)..(12): D-amino acid
  MOD_RES (13)..(13): D-amino acid
IGYHANNSTPPPPC

SEQ ID NO: 17 - protein, 16 aa, Artificial Sequence
  Description of Artificial Sequence Synthetic peptide
  MOD_RES (1)..(1): L-Norleucine
  MOD_RES (12)..(12): D-amino acid
  MOD_RES (13)..(13): D-amino acid
  MOD_RES (14)..(14): D-amino acid
  MOD_RES (15)..(15): D-amino acid
LSLLTEVETTTPPPPC

SEQ ID NO: 18 - protein, 16 aa, Artificial Sequence
  Description of Artificial Sequence Synthetic peptide
  MOD_RES (2)..(2): D-amino acid
  MOD_RES (3)..(3): D-amino acid
  MOD_RES (4)..(4): D-amino acid
  MOD_RES (5)..(5): D-amino acid
  MOD_RES (8)..(8): L-Norleucine
CPPPPTTLSLLTEVET

SEQ ID NO: 19 - protein, 14 aa, Artificial Sequence
  Description of Artificial Sequence Synthetic peptide
  MOD_RES (10)..(10): D-amino acid
  MOD_RES (11)..(11): D-amino acid
  MOD_RES (12)..(12): D-amino acid
  MOD_RES (13)..(13): D-amino acid
ILRTQESSSPPPPC

SEQ ID NO: 20 - protein, 14 aa, Artificial Sequence
  Description of Artificial Sequence Synthetic peptide
  MOD_RES (2)..(2): D-amino acid
  MOD_RES (3)..(3): D-amino acid
  MOD_RES (4)..(4): D-amino acid
  MOD_RES (5)..(5): D-amino acid
CPPPPSSILRTQES

SEQ ID NO: 21 - protein, 16 aa, Artificial Sequence
  Description of Artificial Sequence Synthetic peptide
  MOD_RES (12)..(12): D-amino acid
  MOD_RES (13)..(13): D-amino acid
  MOD_RES (14)..(14): D-amino acid
  MOD_RES (15)..(15): D-amino acid
WSSSASHDGSSPPPPC

SEQ ID NO: 22 - protein, 16 aa, Artificial Sequence
  Description of Artificial Sequence Synthetic peptide
  MOD_RES (2)..(2): D-amino acid
  MOD_RES (3)..(3): D-amino acid
  MOD_RES (4)..(4): D-amino acid
  MOD_RES (5)..(5): D-amino acid
CPPPPSSWSSSASHDG

SEQ ID NO: 23 - protein, 15 aa, Artificial Sequence
  Description of Artificial Sequence Synthetic peptide
  MOD_RES (2)..(2): D-amino acid
  MOD_RES (3)..(3): D-amino acid
  MOD_RES (4)..(4): D-amino acid
  MOD_RES (5)..(5): D-amino acid
CPPPPSTHIEESSSY

SEQ ID NO: 24 - protein, 15 aa, Artificial Sequence
  Description of Artificial Sequence Synthetic peptide
  MOD_RES (11)..(11): D-amino acid
  MOD_RES (12)..(12): D-amino acid
  MOD_RES (13)..(13): D-amino acid
  MOD_RES (14)..(14): D-amino acid
HIEESSSYTSPPPPC

SEQ ID NO: 25 - protein, 18 aa, Artificial Sequence
  Description of Artificial Sequence Synthetic peptide
  MOD_RES (2)..(2): D-amino acid
  MOD_RES (3)..(3): D-amino acid
  MOD_RES (4)..(4): D-amino acid
  MOD_RES (5)..(5): D-amino acid
CPPPPSTSRDNWKGSNRP

SEQ ID NO: 26 - protein, 18 aa, Artificial Sequence
  Description of Artificial Sequence Synthetic peptide
  MOD_RES (14)..(14): D-amino acid
  MOD_RES (15)..(15): D-amino acid
  MOD_RES (16)..(16): D-amino acid
  MOD_RES (17)..(17): D-amino acid
SRDNWKGSNRPSTPPPPC

SEQ ID NO: 27 - protein, 22 aa, Artificial Sequence
  Description of Artificial Sequence Synthetic peptide
  MOD_RES (2)..(2): D-amino acid
  MOD_RES (3)..(3): D-amino acid
  MOD_RES (4)..(4): D-amino acid
  MOD_RES (5)..(5): D-amino acid
CPPPPSTTDGSASGQASTRILK

SEQ ID NO: 28 - protein, 22 aa, Artificial Sequence
  Description of Artificial Sequence Synthetic peptide
  MOD_RES (18)..(18): D-amino acid
  MOD_RES (19)..(19): D-amino acid
  MOD_RES (20)..(20): D-amino acid
  MOD_RES (21)..(21): D-amino acid
TDGSASGQASTRILKTSPPPPC

SEQ ID NO: 29 - protein, 25 aa, Artificial Sequence
  Description of Artificial Sequence Synthetic peptide
  MOD_RES (2)..(2): D-amino acid
  MOD_RES (3)..(3): D-amino acid
  MOD_RES (4)..(4): D-amino acid
  MOD_RES (5)..(5): D-amino acid
CPPPPSSDQVRESRNPGNAEIEDLI

SEQ ID NO: 30 - protein, 25 aa, Artificial Sequence
  Description of Artificial Sequence Synthetic peptide
  MOD_RES (21)..(21): D-amino acid
  MOD_RES (22)..(22): D-amino acid
  MOD_RES (23)..(23): D-amino acid
  MOD_RES (24)..(24): D-amino acid
DQVRESRNPGNAEIEDLISTPPPPC

SEQ ID NO: 31 - protein, 24 aa, Artificial Sequence
  Description of Artificial Sequence Synthetic peptide
  MOD_RES (2)..(2): D-amino acid
  MOD_RES (3)..(3): D-amino acid
  MOD_RES (4)..(4): D-amino acid
  MOD_RES (5)..(5): D-amino acid
CPPPPSSFLARSALILRGSVAHKS

SEQ ID NO: 32 - protein, 24 aa, Artificial Sequence
  Description of Artificial Sequence Synthetic peptide
  MOD_RES (20)..(20): D-amino acid
  MOD_RES (21)..(21): D-amino acid
  MOD_RES (22)..(22): D-amino acid
  MOD_RES (23)..(23): D-amino acid
FLARSALILRGSVAHKSSTPPPPC

SEQ ID NO: 33 - protein, 26 aa, Artificial Sequence
  Description of Artificial Sequence Synthetic peptide
  MOD_RES (2)..(2): D-amino acid
  MOD_RES (3)..(3): D-amino acid
  MOD_RES (4)..(4): D-amino acid
  MOD_RES (5)..(5): D-amino acid
  MOD_RES (9)..(9): L-Norleucine
  MOD_RES (15)..(15): Para-nitro-L-Phenylalanine
CPPPPSSHLAIIKKFTSGRQEKNPSL

SEQ ID NO: 34 - protein, 26 aa, Artificial Sequence
  Description of Artificial Sequence Synthetic peptide
  MOD_RES (2)..(2): L-Norleucine
  MOD_RES (8)..(8): Para-nitro-L-Phenylalanine
  MOD_RES (22)..(22): D-amino acid
  MOD_RES (23)..(23): D-amino acid
  MOD_RES (24)..(24): D-amino acid
  MOD_RES (25)..(25): D-amino acid
HLAIIKKFTSGRQEKNPSLSTPPPPC

SEQ ID NO: 35 - protein, 23 aa, Artificial Sequence
  Description of Artificial Sequence Synthetic peptide
  MOD_RES (2)..(2): D-amino acid
  MOD_RES (3)..(3): D-amino acid
  MOD_RES (4)..(4): D-amino acid
  MOD_RES (5)..(5): D-amino acid
  MOD_RES (9)..(9): L-Norleucine
  MOD_RES (12)..(12): L-Norleucine
  MOD_RES (13)..(13): L-Norleucine
  MOD_RES (15)..(15): L-Norleucine
  MOD_RES (17)..(17): Para-nitro-L-Phenylalanine
CPPPPSTRLKWLLALKFPITADK

SEQ ID NO: 36 - protein, 23 aa, Artificial Sequence
  Description of Artificial Sequence Synthetic peptide
  MOD_RES (2)..(2): L-Norleucine
  MOD_RES (5)..(5): L-Norleucine
  MOD_RES (6)..(6): L-Norleucine
  MOD_RES (8)..(8): L-Norleucine
  MOD_RES (19)..(19): D-amino acid
  MOD_RES (20)..(20): D-amino acid
  MOD_RES (21)..(21): D-amino acid
  MOD_RES (22)..(22): D-amino acid
RLKWLLALKYPITADKSTPPPPC

SEQ ID NO: 37 - protein, 24 aa, Artificial Sequence
  Description of Artificial Sequence Synthetic peptide
  MOD_RES (2)..(2): D-amino acid
  MOD_RES (3)..(3): D-amino acid
  MOD_RES (4)..(4): D-amino acid
  MOD_RES (5)..(5): D-amino acid
  MOD_RES (11)..(11): L-Norleucine
CPPPPSSVAYLLERELVRKTRFLP

SEQ ID NO: 38 - protein, 24 aa, Artificial Sequence
  Description of Artificial Sequence Synthetic peptide
  MOD_RES (3)..(3): Para-nitro-L-Phenylalanine
  MOD_RES (4)..(4): L-Norleucine
  MOD_RES (20)..(20): D-amino acid
  MOD_RES (21)..(21): D-amino acid
  MOD_RES (22)..(22): D-amino acid
  MOD_RES (23)..(23): D-amino acid
VAFLLERELVRKTRFLPSTPPPPC

SEQ ID NO: 39 - protein, 25 aa, Artificial Sequence
  Description of Artificial Sequence Synthetic peptide
  MOD_RES (2)..(2): D-amino acid
  MOD_RES (3)..(3): D-amino acid
  MOD_RES (4)..(4): D-amino acid
  MOD_RES (5)..(5): D-amino acid
  MOD_RES (16)..(16): Para-nitro-L-Phenylalanine
CPPPPSSVAGGTSSVFIEVLHLTQG

SEQ ID NO: 40 - protein, 25 aa, Artificial Sequence
  Description of Artificial Sequence Synthetic peptide
  MOD_RES (9)..(9): Para-nitro-L-Phenylalanine
  MOD_RES (21)..(21): D-amino acid
  MOD_RES (22)..(22): D-amino acid
  MOD_RES (23)..(23): D-amino acid
  MOD_RES (24)..(24): D-amino acid
VAGGTSSVFIEVLHLTQGTSPPPPC

SEQ ID NO: 41 - protein, 25 aa, Artificial Sequence
  Description of Artificial Sequence Synthetic peptide
  MOD_RES (2)..(2): D-amino acid
  MOD_RES (3)..(3): D-amino acid
  MOD_RES (4)..(4): D-amino acid
  MOD_RES (5)..(5): D-amino acid
CPPPPTSYITRNQPEWFRNVLSIAP

SEQ ID NO: 42 - protein, 25 aa, Artificial Sequence
  Description of Artificial Sequence Synthetic peptide
  MOD_RES (1)..(1): Para-nitro-L-Phenylalanine
  MOD_RES (21)..(21): D-amino acid
  MOD_RES (22)..(22): D-amino acid
  MOD_RES (23)..(23): D-amino acid
  MOD_RES (24)..(24): D-amino acid
FITRNQPEWFRNVLSIAPSTPPPPC

SEQ ID NO: 43 - protein, 26 aa, Artificial Sequence
  Description of Artificial Sequence Synthetic peptide
  MOD_RES (2)..(2): D-amino acid
  MOD_RES (3)..(3): D-amino acid
  MOD_RES (4)..(4): D-amino acid
  MOD_RES (5)..(5): D-amino acid
  MOD_RES (9)..(9): L-Norleucine
  MOD_RES (14)..(14): L-Norleucine
  MOD_RES (22)..(22): L-Norleucine
CPPPPSSILFSNKLARLGKGYLFESK

SEQ ID NO: 44 - protein, 26 aa, Artificial Sequence
  Description of Artificial Sequence Synthetic peptide
  MOD_RES (2)..(2): L-Norleucine
  MOD_RES (7)..(7): L-Norleucine
  MOD_RES (15)..(15): L-Norleucine
  MOD_RES (22)..(22): D-amino acid
  MOD_RES (23)..(23): D-amino acid
  MOD_RES (24)..(24): D-amino acid
  MOD_RES (25)..(25): D-amino acid
ILFSNKLARLGKGYLFESKSTPPPPC

SEQ ID NO: 45 - protein, 28 aa, Artificial Sequence
  Description of Artificial Sequence Synthetic peptide
  MOD_RES (2)..(2): D-amino acid
  MOD_RES (3)..(3): D-amino acid
  MOD_RES (4)..(4): D-amino acid
  MOD_RES (5)..(5): D-amino acid
  MOD_RES (13)..(13): L-Norleucine
CPPPPTTDLEALLEWLKTRPILSPLTKG

SEQ ID NO: 46 - protein, 28 aa, Artificial Sequence
  Description of Artificial Sequence Synthetic peptide
  MOD_RES (6)..(6): L-Norleucine
  MOD_RES (24)..(24): D-amino acid
  MOD_RES (25)..(25): D-amino acid
  MOD_RES (26)..(26): D-amino acid
  MOD_RES (27)..(27): D-amino acid
DLEALLEWLKTRPILSPLTKGTTPPPPC

SEQ ID NO: 47 - protein, 28 aa, Artificial Sequence
  Description of Artificial Sequence Synthetic peptide
  MOD_RES (2)..(2): D-amino acid
  MOD_RES (3)..(3): D-amino acid
  MOD_RES (4)..(4): D-amino acid
  MOD_RES (5)..(5): D-amino acid
  MOD_RES (28)..(28): Para-nitro-L-Phenylalanine
CPPPPTSILGFVFTLTVPSERGLQRRRF

SEQ ID NO: 48 - protein, 28 aa, Artificial Sequence
  Description of Artificial Sequence Synthetic peptide
  MOD_RES (24)..(24): D-amino acid
  MOD_RES (25)..(25): D-amino acid
  MOD_RES (26)..(26): D-amino acid
  MOD_RES (27)..(27): D-amino acid
ILGFVFTLTVPSERGLQRRRFTSPPPPC

SEQ ID NO: 49 - protein, 20 aa, Artificial Sequence
  Description of Artificial Sequence Synthetic peptide
  MOD_RES (2)..(2): D-amino acid
  MOD_RES (3)..(3): D-amino acid
  MOD_RES (4)..(4): D-amino acid
  MOD_RES (5)..(5): D-amino acid
  MOD_RES (15)..(15): L-Norleucine
  MOD_RES (19)..(19): L-Norleucine
CPPPPTTLQAYQKRLGVQLR

SEQ ID NO: 50 - protein, 20 aa, Artificial Sequence
  Description of Artificial Sequence Synthetic peptide
  MOD_RES (8)..(8): L-Norleucine
  MOD_RES (12)..(12): L-Norleucine
  MOD_RES (16)..(16): D-amino acid
  MOD_RES (17)..(17): D-amino acid
  MOD_RES (18)..(18): D-amino acid
  MOD_RES (19)..(19): D-amino acid
LQAYQKRLGVQLRTTPPPPC

SEQ ID NO: 51 - protein, 31 aa, Artificial Sequence
  Description of Artificial Sequence Synthetic polypeptide
  MOD_RES (2)..(2): D-amino acid
  MOD_RES (3)..(3): D-amino acid
  MOD_RES (4)..(4): D-amino acid
  MOD_RES (5)..(5): D-amino acid
  MOD_RES (28)..(28): Para-nitro-L-Phenylalanine
CPPPPTTIIGILHLILWILDRLFFKSIFRLF

SEQ ID NO: 52 - protein, 31 aa, Artificial Sequence
  Description of Artificial Sequence Synthetic polypeptide
  MOD_RES (16)..(16): Para-nitro-L-Phenylalanine
  MOD_RES (27)..(27): D-amino acid
  MOD_RES (28)..(28): D-amino acid
  MOD_RES (29)..(29): D-amino acid
  MOD_RES (30)..(30): D-amino acid
IIGILHLILWILDRLFFKSIYRLFTTPPPPC

SEQ ID NO: 53 - protein, 20 aa, Artificial Sequence
  Description of Artificial Sequence Synthetic peptide
  MOD_RES (2)..(2): D-amino acid
  MOD_RES (3)..(3): D-amino acid
  MOD_RES (4)..(4): D-amino acid
  MOD_RES (5)..(5): D-amino acid
CPPPPTTAADLKSTQEAING

SEQ ID NO: 54 - protein, 20 aa, Artificial Sequence
  Description of Artificial Sequence Synthetic peptide
  MOD_RES (16)..(16): D-amino acid
  MOD_RES (17)..(17): D-amino acid
  MOD_RES (18)..(18): D-amino acid
  MOD_RES (19)..(19): D-amino acid
AADLKSTQEAINGTTPPPPC

SEQ ID NO: 55 - protein, 5 aa, Artificial Sequence
  Description of Artificial Sequence Synthetic peptide
  MOD_RES (2)..(2): D-amino acid
  MOD_RES (3)..(3): D-amino acid
  MOD_RES (4)..(4): D-amino acid
  MOD_RES (5)..(5): D-amino acid
CPPPP

SEQ ID NO: 56 - protein, 5 aa, Artificial Sequence
  Description of Artificial Sequence Synthetic peptide
  MOD_RES (1)..(1): D-amino acid
  MOD_RES (2)..(2): D-amino acid
  MOD_RES (3)..(3): D-amino acid
  MOD_RES (4)..(4): D-amino acid
PPPPC

(19) United States
(12) Patent Application Publication
(10) Pub. No.: US 2023/0270838 A1
(43) Pub. Date: Aug. 31, 2023

(54) VACCINE COMPOSITIONS FOR INFLUENZA VIRUSES AND METHODS OF USE

(71) Applicants: Richard ASCIONE, Jersey City, NJ (US); Shengmei QI, Jersey City, NJ (US)

(72) Inventors: Richard ASCIONE, Jersey City, NJ (US); Shengmei QI, Jersey City, NJ (US)

(21) Appl. No.: 18/040,183

(22) PCT Filed: Jul. 30, 2021
(86) PCT No.: PCT/US2021/071080

§ 371 Date: Feb. 1, 2023

Related U.S. Application Data
(60) Provisional application No. 63/060,071, filed on Aug. 2, 2020.

(51) Int. Cl. A61K 39/145; A61K 39/39; A61P 31/16

(52) U.S. Cl. CPC A61K 39/145; A61K 39/39; A61P 31/16; A61K 2039/6037

(57) ABSTRACT

The invention provides pan-influenza vaccine compositions (i.e., vaccine compositions useful against multiple influenza viruses such as H1N1, H2N2, H5N2, etc.), a vaccination regimen for immunization against such influenza diseases, and its use in medicine and in augmenting immune responses to various antigens present in such viruses and to methods of preparation of such compositions. In particular, the invention relates to polyvalent multi-targeting immunogenic compositions comprising influenza viral antigens or antigen preparations thereof from multiple strains associated with human pandemic outbreaks in combination with accessory delivery vehicle(s) and adjuvants.

### CROSS REFERENCE TO RELATED APPLICATIONS

The present application is a U.S. National Phase application under 35 U.S.C. § 371 of International Patent Application No. PCT/US2021/071080 filed Jul. 30, 2021, which claims priority to U.S. Provisional Patent Application No. 63/060,071, filed on Aug. 2, 2020, the entire contents of each of which are herein incorporated by reference.

### SEQUENCE LISTING

The instant application contains a Sequence Listing which has been submitted electronically in ASCII format and is hereby incorporated by reference in its entirety. Said ASCII copy, created on Jul. 29, 2021, is named 259205_000005_SL.txt and is 34,854 bytes in size.

### TECHNICAL FIELD

The present invention relates to an influenza vaccine formulation and vaccination regimen for immunization against disease and its use in medicine and in augmenting immune responses to various antigens present in influenza viruses and to methods of preparation. In particular the invention relates to polyvalent influenza immunogen compositions comprising influenza viral antigens or antigen preparations thereof from influenza strains associated with pandemic outbreaks in combination with delivery vehicle and adjuvants.

### BACKGROUND

Influenza viruses cause worldwide epidemics almost every year, with infections ranging from mild respiratory illnesses to those causing major pulmonary complications resulting in severe morbidity and significant deaths, particularly in the elderly and very young individuals; especially those immuno-compromised as well as those with underlying chronic diseases. Influenza vaccinations play a critical role, primarily in controlling and preventing major epidemic outbreaks and current technology currently consists of employing inactivated virus as vaccine or using the somewhat less effective (K. A. McLean et.al., “The 2015 Global Production Capacity of Seasonal and Pandemic Influenza Vaccine”, Vaccine 2016; 34 (45), 5410-5413) live attenuated virus as vaccines are typically prepared in embryonated chicken eggs. Presently inactivated vaccines are of three forms for antigen preparation: inactivated whole virions; sub-virions where purified virus particles are from disrupted virion particles (split-virion vaccines) or those employing purified hemagglutinin (HA) and neuraminidase (NA) proteins (sub-unit vaccines).

Influenza viruses evolve rapidly by undergoing rapid antigen variation continuously. This antigenic drift affects the function and structure of the outer surface of the viral proteins enabling the virus to escape the hosts' immune surveillance mechanisms and necessitates the nearly annual reformulation of viral preparations for use in these seasonal vaccinations. At unpredictable intervals (four times in the past 100 years, in fact), completely novel influenza viruses emerge having key surface antigens altered, such that totally different subtypes are acquired from prior infectious agents and from various animal and human strains in circulation elsewhere. Likely this pandemic emergence is thought to be due to the segmented viral genomes of influenza viruses that enables radical antigenic variations to appear due to re-assortment mechanisms occurring possibly as a consequence of randomized mixing of multiple segments of viral genomes infecting a single cell simultaneously. These unpredictable, resultant shifted and altered antigens, particularly those located on the outer, receptor interactive surface of the influenza viruses, enable these emerging flu viruses to escape from inhibitory antibodies that were generated previously, which had in prior times enabled transient immunity against those un-reassorted influenza viruses in circulation. Hence these novel re-assorted variants are able to establish a pandemic strain of virus, such as that exemplified by the 2009 H1N1 (“swine flu”) virus that quickly became a pandemic flu in world-wide circulation. In fact, the 2009 pandemic H1N1 virus shared antigenically common features of the pandemic H1N1 virus that afflicted the world's population almost 100 years prior in 1918; fortunately, with seemingly less morbidity and mortality in 2009 than back then, but having an atypical manifestation of having a higher morbidity among younger infected populations.

Emergence of worldwide influenza pandemics, even though infrequent, are nevertheless severe and tend to be devastating in terms of mortality and morbidity especially in undeveloped countries where vaccines and antiviral resources are limited. Pandemic influenzas, unlike the seasonal variants, are notoriously difficult to prepare by typical vaccine strategies for, because of their unpredictable specific surface antigen sequences. Using current egg-based technologies to produce viral vaccines predicates at least a 6-8 month lead time being required for any pandemic vaccine, in which time the viral disease could likely afflict about a third of any population before becoming available for prophylaxis. This pandemic scenario would arise as a consequence of limitations in current virus production methods requiring multi-millions or even billions of chicken eggs, or even the existing limited complex isolation-containment services required of maintaining sterile animal cell culture facilities for live and/or inactivated virus needed for pandemic future vaccines. Thus, effective pandemic vaccines are needed for long lasting immunity against emerging viruses, even those that might recombine or re-assort with highly lethal strains of other specie influenza viruses like the current H5N1 Avian influenza that has associated with it, an overall lethality of close to 60% for those people infected from direct bird contacts.

The vaccines needed for this type of prophylaxis should, in and of themselves, be safe and harmless to individuals handling and receiving them; use of the pathogen itself would be precluded for vaccine preparations given its highly lethal nature. Frequently such preparations may pose difficulties in yields of viruses, given the highly pathogenic nature of the virus inhibits growth in host embryos or cells, thereby compounding the difficulties in a ready and available supply of sufficient vaccine worldwide.

An alternative approach to afford immunologic protection could be to employ short peptide regions of the pathogenic organism, which would correspond to immunologically identifiable regions in the organism known as epitopes. Such peptide epitopes can be constructed synthetically and combined into a multivalent vaccine that could provoke immunologic protection against a pathogen without the need for utilizing any actual pathogen components that would require strict safety/isolation procedure.

A major difficulty of peptide vaccines, however, is that peptides can be poor immunogens and may require larger carrier proteins to evoke substantial antibody and T-cell responses. Additionally, the peptide vaccines may not often provoke substantial enough immune responses or are of such low affinity and titers that they may be ineffectual at binding to the native pathogen because of their decreased ability to retain a recognizable spatial/stable conformation for substantial periods of time to evoke a proper immune response or even to have that immune response access and bind to the natural pathogenic epitope. An advantage to working with peptide vaccines, however, is that they can be synthesized quickly and can be directed towards epitopes that are not normally frequently mutated and/or immunodominant, as occurs frequently in using the native whole virus or pathogen as a vaccine component, thereby allowing escape from immune protection elicited by prior infections as is well known for the influenza group of viruses, the dengue viruses, and retroviruses in general.

### BRIEF SUMMARY OF THE INVENTION

As specified in the Background Section, there is a great need in the art to identify technologies for vaccine compositions designed to provide immunity against a variety of influenza viruses, including variants of existing viruses such as the H1N1, H2N2, and H5N2 viruses, and use this understanding to develop novel vaccine compositions and methods of administering same. The present invention satisfies this and other needs. Embodiments of the present invention relate generally to vaccine compositions comprising peptide epitopes that are common to many influenza viruses, and more specifically to vaccine compositions comprising peptide epitopes that are not normally frequently mutated or immunodominant in combination with immunogenic components, such as for example and not limitation, immunogenic carriers, adjuvants, and short peptides covalently joined to the influenza peptides.

In a first aspect, the invention comprises low amounts of influenza virus antigen that is associated with a pandemic strain or would have the potential of associating with a pandemic virus. The suitable antigens are peptides or peptide mimics of select hemagglutinin antigenic (HA) sequences conserved in all of the known, sequenced pandemic influenza viruses (1918) H1; (1957) H2; (1968) H3; and (2009) H1 as well as in the highly pathogenic Avian virus (2007) H5. At least one or more of these peptide epitopes are not located in immunodominant surface regions of the influenza virion known to mutate frequently. Suitably the chosen hemagglutinin antigenic epitopes are mimicked by peptides chemically synthesized by standard GMP solid phase synthesis methods known to the art.

In a second aspect, the suitable antigens are peptides or peptide mimics of select hemagglutinin (HA) antigenic sequences conserved in all of the known, sequenced seasonal influenza viruses both types A and B from H1-H7 and H8-H16. At least one or more of these peptide epitopes are not located in immunodominant surface regions known to mutate frequently. Suitably the hemagglutinin antigenic epitopes are mimetic peptides chemically synthesized by standard GMP solid phase synthesis methods known to the art.

In a third aspect of the invention, a select ubiquitous pore protein segment, a non-hemagglutinin antigenic epitope, or mimic thereof, known to mutate infrequently and that corresponds to a highly conserved nonapeptide extracellular domain of the matrix-2 (M2) virion ion pore protein epitope region is included in compositions of the invention. Suitably this antigenic epitope is a peptide or peptide mimic that is chemically synthesized by standard GMP solid phase synthesis methods known to the art.

In a fourth aspect of this invention, the suitable antigens are peptides or peptide mimics of select non-hemagglutinin antigenic sequences conserved in all of the known, sequenced seasonal influenza viruses both types A and B; with neuraminidases (NA) selected NA Group 1 containing types N1, N4, N5, and N8 and from NA Group 2 containing types N2, N3, N6, N7, and N9 residues is included in compositions of the invention.

At least one or more of these peptide epitopes described herein are not located in immunodominant surface regions known to mutate frequently. Suitably these non-hemagglutinin antigenic epitopes are peptides or peptide mimics chemically synthesized by standard GMP solid phase synthesis methods known to the art.

In a fifth aspect of this invention, the suitable antigens are peptides or peptide mimics of select non-hemagglutinin nucleoprotein (NP) antigenic sequences conserved in all of the known, sequenced seasonal influenza viruses both types A and B and from HA types H1-H7 and H8-H16 and/or also from NA types N1 to N10. At least one or more of these peptide epitopes are not located in immunodominant surface regions known to mutate frequently. Suitably these non-hemagglutinin antigenic epitopes are peptides or peptide mimics chemically synthesized by standard GMP solid phase synthesis methods known to the art.

In a sixth aspect of this invention, the suitable antigens are peptides or peptide mimics of select non-hemagglutinin polymerase type A and/or B (PA/PB) antigenic sequences conserved in all of the known, sequenced seasonal influenza viruses both types A and B and from HA types H1-H7 and H8-H16 and/or also from NA types N1 to N9. At least one or more of these peptide epitopes are not located in immunodominant surface regions known to mutate frequently. Suitably these non-hemagglutinin polymerase antigenic epitopes are peptides, or peptide mimics, chemically synthesized by standard GMP solid phase synthesis methods known to the art.

In a seventh aspect, any of the disclosed compositions can comprise a specific spacer peptide that permits the proper peptide antigen orientation to the immune system following conjugation of peptide antigens-spacer complex onto the specific protein carrier that constitutes the immunogen and enables the sufficient and sustained persistence of said immunogen to effect useful titers of specific epitope targeting antibody. This spacer peptide consists of four or more specific amino acid residues e.g., four or more proline residues, conjugated to a cysteine residue, preferably at the C or N-terminus, depending on the orientation of the antigen epitope desired. Preferably, there are two hydroxy amino acids (e.g., S or T such as SS, TT, TS, or ST) that are conjugated to the opposite terminus from the cysteine residue. Exemplary spacer peptides can have D-enantiomeric forms of one or more of the amino acids. Preferred conjoiner or spacer peptides include SS, TT, TS, or ST conjugated to ppppC (SEQ ID NO: 55; wherein the lowercase amino acids are D-enantiomers, for use at the C-terminal end of the epitopes discussed herein) and Cpppp (SEQ ID NO: 56) conjugated to SS, TT, TS, or ST, wherein the lowercase amino acids are D-enantiomers, for use at the N-terminal end of the epitopes discussed herein). The linkage of the peptide spacer to an immunogenic carrier should be stable under physiologic conditions and should not spatially interfere with the desired epitope in the antigenic peptide. Moreover, the conjoined spacer peptides afford resistance to proteolytic degradation that extends the circulatory life of these therapeutic peptides enhancing immunity and allowing persistence for antigen presenting cell (APC) presentation. Conjugation to the protein carrier in the invention is by any conventional conjugation method known in the art, and using peptide covalent conjugation chemistry methods known to the art.

In an eighth aspect of the invention, at least three or more peptide antigenic epitope immunogens are combined (comprising of two or more HA epitope peptides and at least one M2 epitope peptide) to form a trivalent (or quadrivalent, pentavalent, etc.,) or polyvalent anti-pandemic immunogen. In some embodiments, the vaccine compositions comprise two or more peptide antigenic epitopes, three or more peptide antigenic epitopes, four or more peptide antigenic epitopes, or five or more peptide antigenic epitopes, optionally coupled to immunogenic carriers. In some embodiments, the vaccine compositions comprise at least five or more peptide antigenic epitope immunogens, optionally coupled to immunogenic carriers. Mixing of polyvalent components is based on antibody titers against specific influenza epitope antigens, determined by standard immunological methods known to the art. Nonlimiting exemplary combinations of epitopes described herein include at least 3-5 peptides, coupled to a spacer peptide, comprising amino acid sequences selected from the group consisting of SEQ ID NOs: 1-54, and/or amino acid sequences and/or mimetic sequences comprising:
(i)(a) SEQ ID NOs: 1, 3, 5, 10, 12, 14, 16, 17, 19, 23, 25, 28, 29, 32, 34, 35, 40, 44, 45, 47, 49, 51, and 53; and/or (i)(b) SEQ ID NOs: 2, 4, 6, 8, 9, 11, 13, 15, 18, 20, 21, 24, 26, 27, 30, 31, 33, 36, 37, 38, 39, 42, 46, 50 and 54; and/or
(ii)(a) SEQ ID NOs: 1-16 and/or (ii)(b) 17-28 and/or (ii)(c) 53-54; and/or
(iii)(a) SEQ ID NOs: 19-28, and/or (iii)(b) 33-44; and/or
(iv)(a) SEQ ID NOs: 10, 12, 14, 15, 17, 23, 25, and/or 27 (iv)(b) 15 and 17-28; and/or 17, 23, 25, and 27; and/or
(v)(a) SEQ ID NOs: 1-16 and/or (v)(b) 24 or 25, optionally coupled to immunogenic carriers.

Additional nonlimiting exemplary combinations of epitopes described herein include at least one peptide comprising an amino acid sequence of SEQ ID NOs: 1, 3, 5, 8, 10, 12, 14, 15, 17, 19, 23, 25, 27, 29, 31, 34, 37, 42, 44, 46, 47, 48, 51, and 53.

Additional nonlimiting exemplary combinations of epitopes described herein include at least one peptide comprising an amino acid sequence of SEQ ID NOs: 1-16, and (i) 18-19, and/or (ii) 20-28, and/or (iii) 53-54.

Additional nonlimiting exemplary combinations of epitopes described herein include at least one peptide comprising an amino acid sequence of SEQ ID NOs: 1-16, and (i) 18-19, and/or (ii) 20-28, and/or (iii) 33-40 and/or (iv) 41-44 and/or (v) 53-54.

Additional nonlimiting exemplary combinations of epitopes described herein include at least one peptide comprising an amino acid sequence of SEQ ID NOs: 10, 12, 14, and 15.

Additional nonlimiting exemplary combinations of epitopes described herein include at least one peptide comprising an amino acid sequence of SEQ ID NOs: 23, 24 and/or 25.

Additional nonlimiting exemplary combinations include at least one amino acid sequence selected from the group consisting of SEQ ID NOs: 1-16, optionally coupled to immunogenic carriers, and at least one amino acid sequence selected from the group consisting of SEQ ID NOs: 17-54, optionally coupled to immunogenic carriers.

Additional nonlimiting exemplary combinations include at least 2 amino acid sequences selected from the group consisting of SEQ ID NOs: 1-16, optionally coupled to immunogenic carriers, and at least 2 amino acid sequences selected from the group consisting of SEQ ID NOs: 17-54, optionally coupled to immunogenic carriers.

Additional nonlimiting exemplary combinations include at least 3 amino acid sequences selected from the group consisting of SEQ ID NOs: 1-16, optionally coupled to immunogenic carriers, and at least 3 amino acid sequences selected from the group consisting of SEQ ID NOs: 17-54, optionally coupled to immunogenic carriers.

In a ninth aspect of the invention, the at least one peptide antigenic epitope is combined with a water-in-oil vehicle that forms a stable emulsion for extended periods of time, with or without added specific adjuvant, using standard emulsification techniques known to the art, to form a polypeptide immunogenic composition. Aluminum based adjuvants are commonly used in the art, however, non-aluminum-based adjuvants can also be used.

In a tenth aspect, the inoculation of the immunogenic composition can occur by different routes, known to the art using suitable dosing regimens determined by taking into account the factors well known to the art, including age, weight, sex and medical status of the patient as well as the route chosen for immunogen administration. Non-limiting exemplary administration routes include subcutaneous injection, intramuscular injection, and/or mucosal (e.g., intranasal or sublingual) administration. The specific aspect of this immunogenic composition requires no reformulation of polyvalent components from year to year, but the periodic boosting of the vaccines may be needed at intervals to be determined by serological titers against antigenic epitopes by standard immunological methods known in the art.

In an eleventh aspect, the invention provides for a method that would by using the formulated immunogenic composition(s) prevent, delay, reduce, inhibit or otherwise restrict the influenza infections in clinical applications for such subjects so exposed. More specifically the invention comprises administration to subjects effective dosages of immunogenic composition(s) that contain such conserved non-mutated influenza protein epitopes coupled to spacers, further conjugated to immunogenic carriers thereof, comprising a vaccine to elicit multiple, protective antibody titers that would afford sustained prophylactic defense against pandemic influenza infection.

### BRIEF DESCRIPTION OF THE FIGURES

The accompanying Figures, which are incorporated in and constitute a part of this specification, illustrate several aspects described below.

FIG. 1A-1B shows antibody kinetics in rabbits following three initial injections of flu immunogens. FIG. 1A, left panel: anti-XL-1 titer of rabbits receiving low and high dosages of XL-1/TT immmunogen; middle panel: anti-XL-2 titer of rabbits receiving low and high dosages of XL-2/TT immmnunogen; right panel: anti-XL-4 titer of rabbits receiving low and high dosages of XL-4/TT immunogen. FIG. 1B shows anti-XL-1 titer (left panel), anti-XL-2 (middle panel), and anti-XL-4 titer (right panel) of rabbits receiving low and high doses of trivalent XL-1+XL-2+XL-4 immunogen (at 6 and 10 meg/dose) of mix (mx)) at times indicated (days 0, 14, 28, and 42).

FIG. 2 shows the kinetics of rabbit antibody induction with DT conjugated immunogens (HA-1; HA-2; and M2 epitopes; SEQ ID NOs: 1, 16, and 17, respectively) after an initial series of three injections on days 0, 14, 21. Titers for each immunogen are plotted through day 56.

FIG. 3 shows the kinetics of Balb/c murine titers of Low dose (2.5 μg) (HA1, M2, and HA2; SEQ ID NOs: 1, 16, and 17, respectively) after an initial 2 doses on days 0 and 28. Titers for each DT immunogen are depicted at times indicated.

FIG. 4 shows Balb/c mice survival following three initial administrations of immunogens HA-1 and the mix (HA-1+M2 pore) at low (1.25 μg) and high 5 (μg) dosages. All mice were compared to controls after exposure at day 38 with 5×LD50 H5N1.

FIG. 5 shows the determination of broadly neutralizing immunogens to hemagglutinin (HA) peptides of various viruses. Titers of eleven TT-conjugated immunogens in rabbits were determined after initial series (days 0, 14, 21) of two doses 10 mcg/dose & 25 mcg/dose, antisera harvested at day 143 for HA-ELISA studies (shown in FIG. 6) on avian, swine, equine and recombinant human influenza HA viral protein binding studies.

FIG. 6 shows ELISA binding studies of select immunogen elicited Rabbit antisera targeting HA specific epitopes of peptides P1 (H1N1 human influenza); P2 (H3N2 Swine influenza)); P3 (H5N1 avian influenza); P4 (H7N7 equine influenza).

### DETAILED DESCRIPTION OF THE INVENTION

As specified in the Background Section, there is a great need in the art to identify technologies for vaccine compositions designed to provide immunity against a variety of influenza viruses, including variants of existing viruses such as the H1N1 virus, and use this understanding to develop novel vaccine compositions and methods of administering same. The present invention satisfies this and other needs. Embodiments of the present invention relate generally to vaccine compositions comprising peptide epitopes that are common to many influenza viruses, and more specifically to vaccine compositions comprising peptide epitopes that are not normally frequently mutated or immunodominant in combination with immunogenic components, such as for example and not limitation, immunogenic carriers, adjuvants, and short peptides covalently joined to the influenza peptides.

To facilitate an understanding of the principles and features of the various embodiments of the invention, various illustrative embodiments are explained below. Although exemplary embodiments of the invention are explained in detail, it is to be understood that other embodiments are contemplated. Accordingly, it is not intended that the invention is limited in its scope to the details of construction and arrangement of components set forth in the following description or examples. The invention is capable of other embodiments and of being practiced or carried out in various ways. Also, in describing the exemplary embodiments, specific terminology will be resorted to for the sake of clarity.

It must also be noted that, as used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named. In other words, the terms “a,” “an,” and “the” do not denote a limitation of quantity, but rather denote the presence of “at least one” of the referenced item.

As used herein, the term “and/or” may mean “and,” it may mean “or,” it may mean “exclusive-or,” it may mean “one,” it may mean “some, but not all,” it may mean “neither,” and/or it may mean “both.” The term “or” is intended to mean an inclusive “or.”

Also, in describing the exemplary embodiments, terminology will be resorted to for the sake of clarity. It is intended that each term contemplates its broadest meaning as understood by those skilled in the art and includes all technical equivalents which operate in a similar manner to accomplish a similar purpose. It is to be understood that embodiments of the disclosed technology may be practiced without these specific details. In other instances, well-known methods, structures, and techniques have not been shown in detail in order not to obscure an understanding of this description. References to “one embodiment,” “an embodiment,” “example embodiment,” “some embodiments,” “certain embodiments,” “various embodiments,” etc., indicate that the embodiment(s) of the disclosed technology so described may include a particular feature, structure, or characteristic, but not every embodiment necessarily includes the particular feature, structure, or characteristic. Further, repeated use of the phrase “in one embodiment” does not necessarily refer to the same embodiment, although it may.

As used herein, the term “about” should be construed to refer to both of the numbers specified as the endpoint (s) of any range. Any reference to a range should be considered as providing support for any subset within that range. Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value. Further, the term “about” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Similarly, as used herein, “substantially free” of something, or “substantially pure”, and like characterizations, can include both being “at least substantially free” of something, or “at least substantially pure”, and being “completely free” of something, or “completely pure”.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

Throughout this description, various components may be identified having specific values or parameters, however, these items are provided as exemplary embodiments. Indeed, the exemplary embodiments do not limit the various aspects and concepts of the present invention as many comparable parameters, sizes, ranges, and/or values may be implemented. The terms “first,” “second,” and the like, “primary,” “secondary,” and the like, do not denote any order, quantity, or importance, but rather are used to distinguish one element from another.

It is noted that terms like “specifically,” “preferably,” “typically,” “generally,” and “often” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention. It is also noted that terms like “substantially” and “about” are utilized herein to represent the inherent degree of uncertainty that may be attributed to any quantitative comparison, value, measurement, or other representation.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “50 mm” is intended to mean “about 50 mm.”

It is also to be understood that the mention of one or more method steps does not preclude the presence of additional method steps or intervening method steps between those steps expressly identified. Similarly, it is also to be understood that the mention of one or more components in a composition does not preclude the presence of additional components than those expressly identified.

The materials described hereinafter as making up the various elements of the present invention are intended to be illustrative and not restrictive. Many suitable materials that would perform the same or a similar function as the materials described herein are intended to be embraced within the scope of the invention. Such other materials not described herein can include, but are not limited to, materials that are developed after the time of the development of the invention, for example. Any dimensions listed in the various drawings are for illustrative purposes only and are not intended to be limiting. Other dimensions and proportions are contemplated and intended to be included within the scope of the invention.

As used herein, the term “subject” or “patient” refers to mammals and includes, without limitation, human and veterinary animals. In a preferred embodiment, the subject is human.

The terms “treat” or “treatment” of a state, disorder or condition include: (1) preventing or delaying the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof (in case of maintenance treatment) or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

As used herein the term “therapeutically effective” applied to dose or amount refers to that quantity of a compound or pharmaceutical composition that when administered to a subject for treating (e.g., preventing or ameliorating) a state, disorder or condition, is sufficient to effect such treatment. The “therapeutically effective amount” will vary depending on the compound or bacteria or analogues administered as well as the disease and its severity and the age, weight, physical condition and responsiveness of the mammal to be treated.

The phrase “pharmaceutically acceptable”, as used in connection with compositions of the invention, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

The terms “pharmaceutical carrier” or “pharmaceutically acceptable carrier” refer to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the pharmaceutical carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin.

In the context of the field of medicine, the term “prevent” encompasses any activity which reduces the burden of mortality or morbidity from disease. Prevention can occur at primary, secondary and tertiary prevention levels. While primary prevention avoids the development of a disease, secondary and tertiary levels of prevention encompass activities aimed at preventing the progression of a disease and the emergence of symptoms as well as reducing the negative impact of an already established disease by restoring function and reducing disease-related complications.

In the context of the present invention, the term “vaccine” (also referred to as an immunogenic composition, vaccine composition, polyvalent composition, polyvalent immunogen, immunogenic epitope, or vaccine formulation) refers to a substance that induces anti-influenza immunity or suppresses influenza virus upon inoculation into a subject. Description of the Invention

The present invention deals with the development of effective protective vaccines and vaccine-like immunogens that can target the influenza viruses that have emerged in the past two decades and include the current influenza variant as well as the recent H1N1, H2N2, and H3N2 influenza variants and is based upon selecting peptides that result in production of antibodies that bind to non-mutated, conserved epitopes within certain influenza proteins.

The invention is based upon the selection of short peptides that are in highly conserved hemagglutinin (HA), neuraminidase (NA), nucleocapsid (NP), RNA polymerase subunits (PA, PB1, and PB2) and matrix-2 (M2) protein regions of most all known influenza A type, and B type viruses. Normally such peptide regions (“epitopes”) are poorly immunogenic and thus can be unable to stimulate any immune system to mount any significant, protective antibody responses against such short peptide-epitope targets. However, the inventors have found that it is possible to modify such epitope sequences by chemical synthesis coupling to a unique short peptide spacer and chemically conjugating these components to a carrier protein that renders these peptide epitopes immunostimulatory.

### Compositions of the Invention

In some embodiments, the compositions of the invention comprise a peptide antigenic epitope, two or more peptide antigenic epitopes, three or more peptide antigenic epitopes, four or more peptide antigenic epitopes, or five or more peptide antigenic epitopes, optionally coupled to immunogenic carriers. In some embodiments, the compositions comprise three to five peptide antigenic epitope immunogens, optionally coupled to immunogenic carriers. In some embodiments, the compositions comprise at least three or more peptide antigenic epitope immunogens, optionally coupled to immunogenic carriers. In any of these compositions, the peptide antigenic epitope immunogens comprise multiple epitopes located in one or more of the hemagglutinin (HA), neuraminidase (NA), nucleocapsid (NP), RNA polymerase subunits (PA, PB1, and PB2) and matrix-2 (M2) proteins to form the immunogenic compositions of the invention. In some embodiments, the peptides of the composition are 6 to 50 amino acids in length, preferably from 6 to 30 amino acids in length.

In some embodiments, the peptides of the composition comprise spacer peptides comprising two hydroxy amino acids (e.g., S or T) coupled to a Cpppp (SEQ ID NO: 55) or ppppC (SEQ ID NO: 56) spacer, which is used to couple the viral peptide to an immunogenic carrier. The orientation of the spacer peptide is selected based on the location of the fusion to the immunogenic carrier.

In an embodiment, vaccine compositions according to the present invention comprise one or more peptides comprising SEQ ID NOs: 1-54, optionally coupled to an immunogenic carrier, preferably two or more peptides comprising SEQ ID NOs: 1-54, optionally coupled to an immunogenic carrier, more preferably three or more peptides comprising SEQ ID NOs: 1-54, optionally coupled to an immunogenic carrier, more preferably four or more peptides comprising SEQ ID NOs: 1-54, optionally coupled to an immunogenic carrier, and most preferably five or more comprising SEQ ID NOs: 1-54, optionally coupled to an immunogenic carrier. A vaccine composition according to the present invention can comprise additional ingredients, such as pharmaceutically acceptable carriers, adjuvants, and/or excipients.

In another embodiment, the vaccine compositions comprise at least three peptides comprising SEQ ID NOs: 1-54, optionally coupled to an immunogenic carrier.

Nonlimiting exemplary combinations of epitopes described herein include at least 3-5 peptides comprising amino acid sequences comprising:
(i)(a) SEQ ID NOs: 1, 3, 5, 10, 12, 14, 16, 17, 19, 23, 25, 28, 29, 32, 34, 35, 40, 44, 45, 47, 49, 51, and 53; and/or (i)(b) SEQ ID NOs: 2, 4, 6, 8, 9, 11, 13, 15, 18, 20, 21, 24, 26, 27, 30, 31, 33, 36, 37, 38, 39, 42, 46, 50 and 54; and/or
(ii)(a) SEQ ID NOs: 1-16 and/or (ii)(b) 17-28 and/or (ii)(c) 53-54; and/or
(iii)(a) SEQ ID NOs: 19-28, and/or (iii)(b) 33-44; and/or
(iv)(a) SEQ ID NOs: 10, 12, 14, 15, 17, 23, 25, and/or 27 (iv)(b) 15 and 17-28; and/or 17, 23, 25, and 27; and/or
(v)(a) SEQ ID NOs: 1-16 and/or (v)(b) 24 or 25, optionally coupled to immunogenic carriers.

Additional nonlimiting exemplary combinations of epitopes described herein include at least one peptide comprising an amino acid sequence of SEQ ID NOs: 1, 3, 5, 8, 10, 12, 14, 15, 17, 19, 23, 25, 27, 29, 31, 34, 37, 42, 44, 46, 47, 48, 51, and 53.

Additional nonlimiting exemplary combinations of epitopes described herein include at least one peptide comprising an amino acid sequence of SEQ ID NOs: 1-16, and (i) 18-19, and/or (ii) 20-28, and/or (iii) 53-54.

Additional nonlimiting exemplary combinations of epitopes described herein include at least one peptide comprising an amino acid sequence of SEQ ID NOs: 1-16, and (i) 18-19, and/or (ii) 20-28, and/or (iii) 33-40 and/or (iv) 41-44 and/or (v) 53-54.

Additional nonlimiting exemplary combinations of epitopes described herein include at least one peptide comprising an amino acid sequence of SEQ ID NOs: 10, 12, 14, and 15.

Additional nonlimiting exemplary combinations of epitopes described herein include at least one peptide comprising an amino acid sequence of SEQ ID NOs: 23, 24 and/or 25.

Additional nonlimiting exemplary combinations include at least one amino acid sequences selected from the group consisting of SEQ ID NOs: 1-16, optionally coupled to immunogenic carriers, and at least one amino acid sequences selected from the group consisting of SEQ ID NOs: 17-54, optionally coupled to immunogenic carriers.

Additional nonlimiting exemplary combinations include at least 2 amino acid sequences selected from the group consisting of SEQ ID NOs: 1-16, optionally coupled to immunogenic carriers, and at least 2 amino acid sequences selected from the group consisting of SEQ ID NOs: 17-54, optionally coupled to immunogenic carriers.

Additional nonlimiting exemplary combinations include at least 3 amino acid sequences selected from the group consisting of SEQ ID NOs: 1-16, optionally coupled to immunogenic carriers, and at least 3 amino acid sequences selected from the group consisting of SEQ ID NOs: 17-54, optionally coupled to immunogenic carriers.

In the following amino acid sequences, capital letters represent L-isomer amino acids and lowercase letters represent D-isomer amino acids, where F* or n-Phe represents para-nitro-phenylalanine—a Y mimetic, and where M* or nL represents L-Norleucine (nLeu), the L-amino acid mimic for L-Methionine. The mimetic amino acids are underlined.
1. An immunogenic composition comprising an Haemagglutinin (HA) mimetic peptide: AINGITNKVNSVIE-SSppppC SEQ ID NO.: 1; optionally coupled to an immunogenic carrier. SEQ ID NO.:1 includes an influenza HA protein peptide mimitope partly homologous to NCBI-listed P03952.2 residues 387-400.
2. An immunogenic composition comprising an HA mimetic peptide: CppppSS-AINGITNKVNSVIE SEQ ID NO.: 2; optionally coupled to an immunogenic carrier. SEQ ID NO.:2 includes an influenza HA protein peptide mimitope partly homologous to NCBI-listed P03952.2 residues 387-400.
3. An immunogenic composition comprising an HA mimetic peptide: RGFFGAIGFIEGGW-TSppppC SEQ ID NO.: 3; optionally coupled to an immunogenic carrier. SEQ ID NO.:3 includes an influenza HA protein peptide mimitope partly homologous to NCBI-listed P03952.2 residues 343-357.
4. An immunogenic composition comprising an HA mimetic peptide: CppppST-RGFFGAIGFIEGGW SEQ ID NO.: 4; optionally coupled to an immunogenic carrier. (SEQ ID NO.:4 includes an influenza HA protein peptide mimitope partly homologous to NCBI-listed P03952.2 residues 343-357.
5. An immunogenic composition comprising an HA mimetic peptide: CppppST-NIHPITIGKSPKYVKS SEQ ID NO.: 5; optionally coupled to an immunogenic carrier. SEQ ID NO.:5 includes an influenza HA protein peptide mimitope partly homologous to NCBI-listed P03952.2 residues 310-325.
6. An immunogenic composition comprising an HA mimetic peptide: NIHPITIGKSPKYVKS-TSppppC SEQ ID NO.: 6; optionally coupled to an immunogenic carrier. (SEQ ID NO.:6 includes an influenza HA protein peptide mimitope partly homologous to NCBI-listed P03952.2 residues 310-325.
7. An immunogenic composition comprising an HA mimetic peptide: CppppSS-nLATGLRNRP SEQ ID NO.: 7; optionally coupled to an immunogenic carrier. SEQ ID NO.:7 includes an influenza HA protein peptide mimitope partly homologous to NCBI-listed P03952.2 residues 330-338.
8. An immunogenic composition comprising an HA mimetic peptide: nLATGLRNRP-SSppppC SEQ ID NO.: 8; optionally coupled to an immunogenic carrier. SEQ ID NO.:8 includes an influenza HA protein peptide mimitope partly homologous to NCBI-listed P03952.2 residues 330-338.
9. An immunogenic composition comprising an HA mimetic peptide: CppppSS-RIENLNKKVDDG SEQ ID NO.: 9; optionally coupled to an immunogenic carrier. SEQ ID NO.:9 includes an influenza HA protein peptide mimitope partly homologous to NCBI-listed P03952.2 residues 419-430.
10. An immunogenic composition comprising an HA mimetic peptide: RIENLNKKVDDG-SSppppC SEQ ID NO.: 10; optionally coupled to an immunogenic carrier. SEQ ID NO.:10 includes an influenza HA protein peptide mimitope partly homologous to NCBI-listed P03952.2 residues 419-430.
11. An immunogenic composition comprising an HA mimetic peptide: CppppSS-DIWTYNAELLV SEQ ID NO.: 11; optionally coupled to an immunogenic carrier. SEQ ID NO.:11 includes an influenza HA protein peptide mimitope partly homologous to NCBI-listed P03952.2 residues 433-443.
12. An immunogenic composition comprising an HA mimetic peptide: DIWTYNAELLV-SSppppC SEQ ID NO.: 12; optionally coupled to an immunogenic carrier. SEQ ID NO.:12 includes an influenza HA protein peptide mimitope partly homologous to NCBI-listed P03952.2 residues 433-443.
13. An immunogenic composition comprising an HA mimetic peptide: CppppSS-EIGNGSFEFYHK SEQ ID NO.: 13; optionally coupled to an immunogenic carrier. SEQ ID NO.:13 includes an influenza HA protein peptide mimitope partly homologous to NCBI-listed P03952.2 residues 475-486.
14. An immunogenic composition comprising an HA mimetic peptide: EIGNGSFEFYHK-SSppppC SEQ ID NO.: 14; optionally coupled to an immunogenic carrier. SEQ ID NO.:14 includes an influenza HA protein peptide mimitope partly homologous to NCBI-listed P03952.2 residues 475-486.
15. An immunogenic composition comprising an HA mimetic peptide: CppppTS-IGYHANNST SEQ ID NO.: 15; optionally coupled to an immunogenic carrier. SEQ ID NO.:15 includes an influenza HA protein peptide mimitope partly homologous to NCBI-listed P03952.2 residues 23-30.
16. An immunogenic composition comprising an HA mimetic peptide: IGYHANN-STppppC SEQ ID NO.: 16; optionally coupled to an immunogenic carrier. SEQ ID NO.:16 includes an influenza HA protein peptide mimitope partly homologous to NCBI-listed P03952.2 residues 23-30.
17. An immunogenic composition comprising an extracellular MATRIX 2 Pore (M2e) mimetic peptide: nLSLLTEVET-TTppppC SEQ ID NO.: 17; optionally coupled to an immunogenic carrier. SEQ ID NO.:17 includes an influenza M2e protein peptide mimitope partly homologous to NCBI-listed ABD59884.1 residues 1-9.
18. An immunogenic composition comprising an extracellular MATRIX 2 Pore (M2e) mimetic peptide: CppppTT-nLSLLTEVET SEQ ID NO.: 18; optionally coupled to an immunogenic carrier. SEQ ID NO.:18 includes an influenza M2e protein peptide mimitope partly homologous to NCBI-listed ABD59884.1 residues 1-9.
19. An ic composition comprising a Neuraminidase (NA) mimetic peptide: ILRTQES-SSppppC SEQ ID NO.: 19; optionally coupled to an immunogenic carrier. SEQ ID NO.:19 includes an influenza NA protein peptide mimitope partly homologous to NCBI-listed YP_009118627 residues 223-229.
20. An immunogenic composition comprising a NA mimetic peptide: CppppSS-ILRTQES SEQ ID NO.: 20; optionally coupled to an immunogenic carrier. SEQ ID NO.:20 includes an influenza NA protein peptide mimitope partly homologous to NCBI-listed YP_009118627 residues 223-229.
21. An immunogenic composition comprising a NA mimetic peptide: WSSSASHDG-SSppppC SEQ ID NO.: 21; optionally coupled to an immunogenic carrier. SEQ ID NO.:21 includes an influenza NA protein peptide mimitope partly homologous to NCBI-listed YP_009118627 residues 179-187.
22. An immunogenic composition comprising a NA mimetic peptide: CppppSS-WSSSASHDG SEQ ID NO.: 22; optionally coupled to an immunogenic carrier. SEQ ID NO.:22 includes an influenza NA protein peptide mimitope partly homologous to NCBI-listed YP_009118627 residues 179-187.
23. An immunogenic composition comprising a NA mimetic peptide: CppppST-HIEESSSY SEQ ID NO.: 23; optionally coupled to an immunogenic carrier. SEQ ID NO.:23 includes an influenza NA protein peptide mimitope partly homologous to NCBI-listed YP_009118627 residues 275-282.
24. An immunogenic composition comprising a NA mimetic peptide: HIEESSSY-TSppppC SEQ ID NO.: 24; optionally coupled to an immunogenic carrier. SEQ ID NO.:24 includes an influenza NA protein peptide mimitope partly homologous to NCBI-listed YP_009118627 residues 275-282.
25. An immunogenic composition comprising a NA mimetic peptide: CppppST-SRDNWKGSNRP SEQ ID NO.: 25; optionally coupled to an immunogenic carrier. SEQ ID NO.:25 includes an influenza NA protein peptide mimitope partly homologous to NCBI-listed YP_009118627 residues 292-302.
26. An immunogenic composition comprising a NA mimetic peptide: SRDNWKGSNRP-STppppC SEQ ID NO.: 26; optionally coupled to an immunogenic carrier. SEQ ID NO.:26 includes an influenza NA protein peptide mimitope partly homologous to NCBI-listed YP_009118627 residues 292-302.
27. An immunogenic composition comprising a NA mimetic peptide: CppppST-TDGSASGQASTRILK SEQ ID NO.: 27; optionally coupled to an immunogenic carrier. SEQ ID NO.:27 includes an influenza NA protein peptide mimitope partly homologous to NCBI-listed YP_009118627 residues 243-257.
28. An immunogenic composition comprising a NA mimetic peptide: TDGSASGQASTRILK-TSppppC SEQ ID NO.: 28; optionally coupled to an immunogenic carrier. SEQ ID NO.:27 includes an influenza NA protein peptide mimitope partly homologous to NCBI-listed YP_009118627 residues 243-257.
29. An immunogenic composition comprising a Nucleoprotein (NP) mimetic peptide: CppppSS-DQVRESRNPGNAEIEDLI SEQ ID NO.: 29; optionally coupled to an immunogenic carrier. SEQ ID NO.:29 includes an influenza NP protein peptide mimitope partly homologous to NCBI-listed ACZ46585.1 residues 240-257.
30. An immunogenic composition comprising a NP mimetic peptide: DQVRESRNPGNAEIEDLI-STppppC SEQ ID NO.: 30; optionally coupled to an immunogenic carrier. SEQ ID NO.:30 includes an influenza NP protein peptide mimitope partly homologous to NCBI-listed ACZ46585.1 residues 240-257.
31. An immunogenic composition comprising a NP mimetic peptide: CppppSS-FLARSALILRGSVAHKS SEQ ID NO.: 31; optionally coupled to an immunogenic carrier. SEQ ID NO.:31 includes an influenza NP protein peptide mimitope partly homologous to NCBI-listed ACZ46585.1 residues 258-274.
32. An immunogenic composition comprising a NP mimetic peptide: FLARSALILRGSVAHKS-STppppC SEQ ID NO.: 32; optionally coupled to an immunogenic carrier. SEQ ID NO.:32 includes an influenza NP protein peptide mimitope partly homologous to NCBI-listed ACZ46585.1 residues 258-274.
33. An immunogenic composition comprising a Polymerase B2 (PB2) mimetic peptide: CppppSS-HnLAIIKKF*TSGRQEKNPSL SEQ ID NO.: 33; optionally coupled to an immunogenic carrier. SEQ ID NO.:33 includes an influenza PB2 protein peptide mimitope partly homologous to NCBI-listed CAZ65595.1 residues 27-45.
34. An immunogenic composition comprising a Polymerase B2 (PB2) mimetic peptide: HnLAIIKKF*TSGRQEKNPSL-STppppC SEQ ID NO.: 34; optionally coupled to an immunogenic carrier. SEQ ID NO.:34 includes an influenza PB2 protein peptide mimitope partly homologous to NCBI-listed CAZ65595.1 residues 27-45.
35. An immunogenic composition comprising a Polymerase B2 (PB2) mimetic peptide: CppppST-RnLKWnLnLAnLKF*PITADK SEQ ID NO.: 35; optionally coupled to an immunogenic carrier. SEQ ID NO.:35 includes an influenza PB2 protein peptide mimitope partly homologous to NCBI-listed CAZ65595.1 residues 46-61.
36. An immunogenic composition comprising a Polymerase B2 (PB2) mimetic peptide: RnLKWnLnLAnLKYPITADK-STppppC SEQ ID NO.: 36; optionally coupled to an immunogenic carrier. SEQ ID NO.:36 includes an influenza PB2 protein peptide mimitope partly homologous to NCBI-listed CAZ65595.1 residues 46-61.
37. An immunogenic composition comprising a Polymerase B2 (PB2) mimetic peptide: CppppSS-VAYnLLEREELVRKTRFLP SEQ ID NO.: 37; optionally coupled to an immunogenic carrier. SEQ ID NO.:37 includes an influenza PB2 protein peptide mimitope partly homologous to NCBI-listed CAZ65595.1 residues 203-219.
38. An immunogenic composition comprising a Polymerase B2 (PB2) mimetic peptide: VAF*nLLER ELVRKTRFLP-STppppC SEQ ID NO.: 38; optionally coupled to an immunogenic carrier. SEQ ID NO.:38 includes an influenza PB2 protein peptide mimitope partly homologous to NCBI-listed CAZ65595.1 residues 203-219.
39. An immunogenic composition comprising a Polymerase B2 (PB2) mimetic peptide: CppppSS-VAGGTSSVF*IEVLHLTQG SEQ ID NO.: 39; optionally coupled to an immunogenic carrier. SEQ ID NO.:39 includes an influenza PB2 protein peptide mimitope partly homologous to NCBI-listed CAZ65595.1 residues 220-237.
40. An immunogenic composition comprising a Polymerase B2 (PB2) mimetic peptide: VAGGTSSVF*IEVLIHLTQG-TSppppC SEQ ID NO.: 40; optionally coupled to an immunogenic carrier. SEQ ID NO.:40 includes an influenza PB2 protein peptide mimitope partly homologous to NCBI-listed CAZ65595.1 residues 220-237.
41. An immunogenic composition comprising a Polymerase B1 (PB1) mimetic peptide: CppppTS-YITRNQPEWFRNVLSIAP SEQ ID NO.: 41; optionally coupled to an immunogenic carrier. SEQ ID NO.:41 includes an influenza PB1 protein peptide mimitope partly homologous to NCBI-listed ABD59820.1 residues 324-342.
42. An immunogenic composition comprising a Polymerase B1 (PB1) mimetic peptide: F*ITRNQPEWFRNVLSIAP-STppppC SEQ ID NO.: 42; optionally coupled to an immunogenic carrier. SEQ ID NO.:42 includes an influenza PB1 protein peptide mimitope partly homologous to NCBI-listed ABD59820.1 residues 324-342.
43. An immunogenic composition comprising a Polymerase B1 (PB1) mimetic peptide: CppppSS-InLFSNKnLARLGKGYnLFESK SEQ ID NO.: 43; optionally coupled to an immunogenic carrier. SEQ ID NO.:43 includes an influenza PB1 protein peptide mimitope partly homologous to NCBI-listed ABD59820.1 residues 343-360.
44. An immunogenic composition comprising a Polymerase B1 (PB1) mimetic peptide: IFSNKnLARLGKGYnLFESK-STppppC SEQ ID NO.: 44; optionally coupled to an immunogenic carrier. SEQ ID NO.:44 includes an influenza PB1 protein peptide mimitope partly homologous to NCBI-listed ABD59820.1 residues 343-360.
45. An immunogenic composition comprising a Matrix 1A (MIA) mimetic peptide: CppppTT-DLEnLEWLKTRPILSPLTKG SEQ ID NO.: 45; optionally coupled to an immunogenic carrier. SEQ ID NO.:45 includes an influenza PB1 protein peptide mimitope partly homologous to NCBI-listed ADB81462.1 residues 36-58.
46. An immunogenic composition comprising a Matrix 1A (MIA) mimetic peptide: DLEALnLEWLKTIRPILSPLTKG-TTppppC SEQ ID NO.: 46; optionally coupled to an immunogenic carrier. SEQ ID NO.:46 includes an influenza PB1 protein peptide mimitope partly homologous to NCBI-listed ADB81462.1 residues 36-58.
47. An immunogenic composition comprising a Matrix 1A (MIA) mimetic peptide: CppppTS-ILGFVFTLTVPSERGLQIRRRF* SEQ ID NO.: 47; optionally coupled to an immunogenic carrier. SEQ ID NO.:47 includes an influenza MIA protein peptide mimitope partly homologous to NCBI-listed ADB81462.1 residues 59-79.
48. An immunogenic composition comprising a Matrix 1A (MIA) mimetic peptide: ILGVFTLTVPSERGLQRRRF-TSppppC SEQ ID NO.: 48; optionally coupled to an immunogenic carrier. SEQ ID NO.:47 includes an influenza MIA protein peptide mimitope partly homologous to NCBI-listed ADB81462.1 residues 59-79.
49. An immunogenic composition comprising a Matrix 1A (MIA) mimetic peptide: CppppTT-LQAYQKRnLGVQnLR SEQ ID NO.: 49; coupled to an immunogenic carrier. SEQ ID NO.:49 includes an influenza MIA protein peptide mimitope partly homologous to NCBI-listed ADB81462.1 residues 237-250.
50. An immunogenic composition comprising a Matrix 1A (M1) mimetic peptide: LQAYQKRnLGVQnLR-TTppppC SEQ ID NO.: 50; coupled to an immunogenic carrier. SEQ ID NO.:50 includes an influenza MIA protein peptide mimitope partly homologous to NCBI-listed ADB81462.1 residues 237-250.
51. An immunogenic composition comprising a Matrix 2 (M2) mimetic peptide: CppppTT-IIGILHLILWILDRLFFKSIF*RLF SEQ ID NO.: 51; optionally coupled to an immunogenic carrier. SEQ ID NO.:51 includes an influenza M2 protein peptide mimitope partly homologous to NCBI-listed ADB59884.1 residues 32-55.
52. An immunogenic composition comprising a Matrix 2 (M2) mimetic peptide: IIGILHLILWILDRLF*FKSIYRLF-TTppppC SEQ ID NO.: 52; optionally coupled to an immunogenic carrier. SEQ ID NO.:52 includes an influenza M2 protein peptide mimitope partly homologous to NCBI-listed ADB59884.1 residues 32-55.
53. An immunogenic composition comprising an HA mimetic T-cell immunogenic peptide: CppppTT-AADLKSTQEAING SEQ ID NO.: 53; optionally coupled to an immunogenic carrier. SEQ ID NO.:53 includes an influenza HA protein peptide mimitope partly homologous to NCBI-listed P03952.2 residues 378-390.
54. An immunogenic composition comprising an HA mimetic T-Cell immunogenic peptide: AADLKSTQEAING-TTppppC SEQ ID NO.: 54; optionally coupled to an immunogenic carrier. SEQ ID NO.:54 includes an influenza HA protein peptide mimitope partly homologous to NCBI-listed P03952.2 residues 378-390. 55. The immunogenic composition comprising at least one peptide comprising an amino acid sequence of SEQ ID NOs: 1-54, wherein the immunogenic carrier is selected from the group consisting of diphtheria toxoid (DT), tetanus toxoid (TT), pertussin toxoid (PT), Pure Protein Derivative (PPD), polio virus and any FDA approved commercial pharmaceutical carrier.
56. The immunogenic composition comprising at least one peptide comprising an amino acid sequence of SEQ ID NOs:1-54, wherein the immunogenic carrier is diphtheria toxoid.
57. A pharmaceutical composition comprising an effective amount of the immunogenic composition of SEQ ID NOs: 1-54 and a pharmaceutically acceptable carrier.
58. The pharmaceutical composition comprising at least one peptide comprising an amino acid sequence of SEQ ID NOs: 1-54 and the pharmaceutically acceptable carrier of item 57, wherein the pharmaceutically acceptable carrier comprises an emulsion of an aqueous phase and an oily phase, wherein the oily phase is an oily vehicle comprising squalene, squalane, Polysorbate 80, Sorbitan monooleate, and Polysorbate 40.
59. The pharmaceutical composition comprising at least one peptide comprising an amino acid sequence of SEQ ID NOs:1-54 and the pharmaceutically acceptable carrier of item 57, wherein the pharmaceutically acceptable carrier comprises an emulsion of an aqueous phase and an oily phase, wherein the oily phase is an oily vehicle comprising squalene, squalane, Polysorbate 80, Sorbitan monooleate, Polysorbate 40, and aluminum monostearate.
60. The pharmaceutical composition comprising at least one peptide comprising an amino acid sequence of claim SEQ ID NOs:1-54 and the pharmaceutically acceptable carrier of item 57, wherein the pharmaceutically acceptable carrier comprises an emulsion of an aqueous phase and an oily phase, wherein the oily phase is an oily vehicle comprising squalene, squalane, Polysorbate 80, Sorbitan monooleate, Polysorbate 40, and wherein either the oily or aqueous phase contains adjuvants comprising, but not limited to: Nor-MDP, Ergamisol, Cimetidine, Praziquantel, Imiquimod, uric acid, cyclic diguanylate, and/or other cyclic dinucleotides that activate STING immunological pathways, synthetic cGAS-STING adjuvants, CpG oligomers, threonyl-N-acetyl-muramyl-L-alanyl-D-isoglutamine, Isoprinosine, mannan, trehalose dimycolate, QS-21 and alpha-galactosylceramide (α-GalCer) or alphaglucosylceramide (α-GluCer).
61. A pharmaceutical composition comprising a therapeutically effective amount of the immunogenic composition comprising at least one peptide comprising an amino acid sequence of SEQ ID NOs: 1-54 and a pharmaceutically acceptable carrier to effect disease protective titers in the majority of hosts vaccinated either intramuscularly, subcutaneously, or mucosally (including intranasally or sublingually).
62. A vaccine composition comprising a therapeutically effective amount of the immunogenic composition comprising at least one peptide comprising an amino acid sequence of SEQ ID NOs: 1-54 and a pharmaceutically acceptable carrier to effect disease protective titers in the majority of hosts vaccinated either intramuscularly, subcutaneously, or mucosally (including intranasally or sublingually).
63. The composition of items 1-62, wherein the at least one peptide comprises an amino acid sequence of SEQ ID NOs: 1, 3, 5, 8, 10, 12, 14, 15, 17, 19, 23, 25, 27, 29, 31, 34, 37, 42, 44, 46, 47, 48, 51, and 53.
64. The composition of items 1-62, wherein the at least one peptide comprises an amino acid sequence of SEQ ID NOs: 1-16, and (i) 18-19, and/or (ii) 20-28, and/or (iii) 53-54.
64. The composition of items 1-62, wherein the at least one peptide comprises an amino acid sequence of SEQ ID NOs: 1-16, and (i) 18-19, and/or (ii) 20-28, and/or (iii) 33-40 and/or (iv) 41-44 and/or (v) 53-54.
65. The composition of items 1-62, wherein the at least one peptide comprises an amino acid sequence of SEQ ID NOs: 10, 12, 14, and 15.
66. The composition of items 1-62, wherein the at least one peptide comprises an amino acid sequence of SEQ ID NOs: 23, 24 and/or 25.
67. The composition of items 1-62, comprising at least two amino acids selected from at least one of the following combinations:
   (i)(a) SEQ ID NOs: 1, 3, 5, 10, 12, 14, 16, 17, 19, 23, 25, 28, 29, 32, 34, 35, 40, 44, 45, 47, 49, 51, and 53; and (i)(b) SEQ ID NOs: 2, 4, 6, 8, 9, 11, 13, 15, 18, 20, 21, 24, 26, 27, 30, 31, 33, 36, 37, 38, 39, 42, 46, 50 and 54; and/or
   (ii)(a) SEQ ID NOs: 1-16 and (ii)(b) 17-28 and/or (ii)(c) 53-54; and/or
   (iii)(a) SEQ ID NOs: 19-28, and/or (iii)(b) 33-44; and/or
   (iv)(a) SEQ ID NOs: 10, 12, 14, 15, 17, 23, 25, and/or 27 (iv)(b) 15 and 17-28; and/or 17, 23, 25, and 27; and/or
   (v)(a) SEQ ID NOs: 1-16 and/or (v)(b) 23, 24 or 25, optionally coupled to an immunogenic carrier.
68. The composition of items 1-62 comprising: (i) at least one peptide comprising an amino acid sequence of SEQ ID NOs: 1-16; and (ii) at least one peptide comprising an amino acid sequence of SEQ ID NOs: 17-54.
69. The composition of items 1-62 comprising: (i) at least two peptides comprising an amino acid sequence of SEQ ID NOs: 1-16; and (ii) at least two peptides comprising an amino acid sequence of SEQ ID NOs: 17-54.
70. The composition of items 1-62 comprising: (i) at least three peptides comprising an amino acid sequence of SEQ ID NOs: 1-16; and (ii) at least three peptides comprising an amino acid sequence of SEQ ID NOs: 17-54.

The invention is able to elicit disease ameliorating and/or protective antibodies and/or T-cell responses able to recognize non-mutated, highly conserved epitope hemagglutinin regions that may be cryptic in the intact virion and not readily recognized by conventional vaccination procedures due to the presence of the more immunodominant and highly mutable surface hemagglutinin epitopes.

The invention is able to elicit disease ameliorating and/or protective antibodies and/or T-cell responses able to recognize non-mutated, highly conserved non-hemagglutinin epitope region that has not been used in conventional whole virus, split virion or subunit influenza viral vaccines, but which is widely present in all type A influenza viruses.

The invention provides immunogens that can elicit high antibody titers, which offer broad protection against infection, particularly across many of the varied types and strains of known influenza viruses. The invention is able to afford immunological protection against pandemic viruses that have not altered the conserved cryptic sites in over 100 years. The invention also is readily and easily modifiable by alteration of easily prepared peptide epitopes and can be used to elicit other antibodies for any number of emerging pathogens that threaten human populations.

The invention describes usage of non-pathogen, non-viral immunogen components that are chemically synthesized by standard GMP chemistries known in the art. These components do not require the use of pathogenic microorganisms, or infected embryonated eggs, or even require cells and complex, sterile media and containment facilities that widely used viral vaccines currently utilize.

The methods required for these non-cellular preparations can result in a purer vaccine preparation with no contaminating microorganisms or viruses that can be introduced using animal cell cultures. Also, the heterogeneity of targeted epitopes, particularly those in the hemagglutinin protein that one experiences using live chicken embryonated viral products (Rajakumar et al, PNAS (1990) 87:4154-4158) is precluded by this invention. In the absence of live viruses as such there is no need to employ denaturation agents or organic solvents to kill and inactivate or extract virus material for vaccines and thus the invention avoids denaturing antigenic epitopes or other items of safety concerns arising from the use of such chemicals that would remain as residuals. Individuals with egg allergies and chemical sensitivities (e.g., mercuric containing compounds) would not have such materials to contend with in these peptide vaccines that contain no live cellular or viral components and thus avoid hypersensitivity issues that arise with commonly used viral vaccines in current commercial production.

The invention provides a broadly protective pan-influenza vaccine that would be protective for all pandemic viral strains thus far known over the last 100 years; one that is easily and rapidly manufactured from readily obtainable peptide materials without the need for live cell or virus materials and is able to yield a highly purified product that would be considerably more cost efficient to produce due to the absence of containment facilities that commercial vaccines produced in avian eggs or in live animal cells currently require.

The invention employs the administration of multi-valent influenza, highly conserved mimic epitopes (mimitopes) in a formulated immunogen designed to induce broad prophylactic immunization in the host animal or human, following initial series of one or more administrations of such immunogen either by subcutaneous, intramuscular, aerosol/inhaled or mucosal/intranasal routes as required. By immunization the inventors refer to a process whereby the host induces an innate immune response initially followed by an adaptive response of continual protective levels of humoral antibody and T-cell responses directed against targeted regions of multiple influenza type A & B proteins, such as the matrix pore proteins, the basic RNA dependent viral RNA-polymerases, the nuclear capsid protein, the neuraminidase proteins and the haemagglutinin proteins. By immunogen the inventors refer to multi-antigen containing composition suspended in a nano-sized-emulsion vehicle (oil/water or water/oil) designed to evoke an immunization response in animal and/or human hosts to provide protection against specific diseases.

The inventors have discovered that modified amino acids in epitopic regions (referred to herein as mimic-epitopes or “mimitopes” that) can be used to enhance the recognition of “foreign” antigens and therefore activate intrinsic and adaptive immune systems accordingly to engender greater anti-viral immune responses. More specifically it is recognized that our natural innate immune system in its processing by neutrophils, monocytes and macrophages (collectively known as antigen presentation cells (APCs)) are able to modify the microbial antigens by peroxidation, halogenation and nitrosylation reactions. Such innate immune chemical modifications are known by the art to enhance and augment the antigenicity and subsequent immunogenicity of the microbial antigens.

Accordingly, the present invention includes and encompasses the employment of one or more halogenated or nitrogenated amino acid residues, such as para-nitro-phenylalanine or para-chloro-phenylalanine to augment the immunogenicity of the immunogen. For example, para-nitrophenylalanine (nPhe/F*) is listed in certain of the inventive mimic epitope residues (mimitopes) of tyrosine (Y) (e.g., Seq ID NOs: 33, No:34, No:35, No:38, No:39, No:40, No:42, No:47, No:51 and No:52) detailed herein. Similarly, certain of the inventive immunogenic epitopes contain in their residues mimetics of methionine (M) as the amino acid, Norleucine (nL) (e.g., Seq ID NOs: 7; 36; 37; 38; 43; 44; 45, 46, 51, and 52.

The inventors have discovered that certain smaller peptide mimetics (typically less than 30 amino acid residues) would be able to enhance their recognition by APCs, by spatially orienting themselves especially with more resistant residues to proteolysis; likely allowing a longer period for antigen presentations. Therefore, the invention includes a series of D-amino acid residue as spacers placed adjacent to epitopes in the mimitopes in all 54 of the listed sequences herein

According to one aspect of the invention, therefore, the improved mimitope immunogens are able to generate multitargeting polyclonal antibodies against viral receptor binding domain and distal stalk regions and their other type A and B influenza protein species, respectively.

Illustrative of such immunogens is one that comprises (i) a preferred peptide of the haemagglutinin (HA) amino acid residues: an immunogenic composition comprising a protein mimetic peptide: Ala-Ile-Asp-Gly-Ile-Thr-Asn-Lys-Val-Asn-Ser-Val-Ile-Glu-Ser-Ser-pro-pro-pro-pro-Cys) (amino acid residues 387-400 of the influenza HA Protein P03452 (SEQ ID NO.: 1); coupled to an immunogenic carrier; optionally coupled to (ii) an immunogenic carrier. Accordingly, this embodiment incorporates a haemagglutinin (HA) protein stalk component mimetic epitope (mimitope) plus a 7 amino-acid C-terminal spacer to constitute an immunomimic that is a 21 amino-acid peptide.

Similarly, another preferred embodiment incorporates an influenza HA stalk protein component mimitope peptide: Arg-Gly-Phe-Phe-Gly-Ala-Ile-Gly-Phe-Ile-Glu-Gly-Gly-Trp-Thr-Ser-pro-pro-pro-pro-Cys (residues 343-357 of HA Protein P03452) plus a C-terminal 7 amino-acid spacer (SEQ ID NO.:3); to constitute an immunomimic that is a 21 amino-acid peptide; optionally coupled to an immunogenic carrier.

In accordance with the above, another further aspect of the invention, is an improved immunogen that can generate polyclonal antibodies against the preferred consensus epitope sequences of the influenza HA protein, listed as P03452. Illustrative of these immunogens are ones that comprises an HA stalk region peptide of the preferred sequence: Cys-pro-pro-pro-pro-Ser-Thr-Asn-Ile-His-Pro-Ile-Thr-Ile-Gly-Lys-Ser-Pro-Lys-Tyr-Val-Lys-Ser Cys (amino acid residues 310-325 of HA Protein P03452) plus a N-terminal 7 amino-acid spacer (SEQ ID NO: 5); optionally coupled to an immunogenic carrier, as described herein. Again, the non-toxic, more immunogenic diphtheria or tetanus toxoid protein is a preferred immunogenic carrier for this preferred embodiment.

A further embodiment of this invention is another preferred immunomimic that comprises a mimetic peptide of the influenza HA stalk sequence: Arg-Ile-Glu-Asn-Leu-Asn-Lys-Lys-Val-Asp-Asp-Gly-Ser-Ser-pro-pro-pro-pro-Cys (amino acid residues 419-430 of HA Protein P03452) plus a C-terminal 7 amino-acid spacer (SEQ ID NO. 10), optionally coupled to an immunogenic carrier, as described herein. Again, the non-toxic, more immunogenic diphtheria or tetanus toxoid protein is a preferred immunogenic carrier in this embodiment.

As an additional embodiment is another preferred immunomimic that comprises a mimetic peptide of the influenza HA stalk sequence: Asp-Ile-Trp-Thr-Tyr-Asn-Ala-Glu-Leu-Leu-Val-Ser-Ser-pro-pro-pro-pro-Cys (amino acid residues 433-443 of HA Protein P03452) plus a C-terminal 7 amino-acid spacer (SEQ ID NO. 12), optionally coupled to an immunogenic carrier, as described herein. The non-toxic, diphtheria or tetanus toxoid protein is a preferred immunogenic carrier for this preferred embodiment.

Still another preferred embodiment of the invention is an immunomimic peptide of the influenza HA stalk sequence: Glu-Ile-Gly-Asn-Gly-Ser-Phe-Glu-Phe-Tyr-His-Lys-Ser-Ser-pro-pro-pro-pro-Cys (amino acid residues 475-486 of HA Protein P03452) plus a C-terminal 7 amino-acid spacer (SEQ ID NO. 14), optionally coupled to an immunogenic carrier, as described herein. Again, the non-toxic immunogenic diphtheria or tetanus toxoid protein is a preferred immunogenic carrier for this preferred embodiment.

Yet another preferred embodiment of the invention is an immunomimic peptide of the influenza HA receptor region sequence: Cys-pro-pro-pro-pro-Thr-Ser-Ile-Gly-Tyr-His-Ala-Asn-Asn (amino acid residues 23-30 of HA Protein P03452); plus a N-terminal spacer (SEQ ID NO.: 15), optionally coupled to an immunogenic carrier, as described herein. Again, the non-toxic, more immunogenic diphtheria or tetanus toxoid protein is a preferred immunogenic carrier for this preferred embodiment.

Another embodiment of the invention is a preferred immunomimic peptide that comprises an influenza Matrix 2 pore mimetic peptide of the sequence: nLeu-Ser-Leu-Leu-Thr-Glu-Val-Glu-Thr-Thr-Thr-pro-pro-pro-pro-Cys (amino acid residues 1-9 of influenza A virus M2 Protein DQ415351.1); plus a C-terminal spacer (SEQ ID NO.: 17) optionally coupled to a preferred immunogenic carrier, as described herein. Accordingly, this mimic represents an improved immunogen able to generate polyclonal antibodies against the preferred consensus amino terminal epitope sequence of the influenza Matrix-2 pore protein.

Also, an embodiment of the invention is a preferred immunomimic that comprises an influenza neuraminidase peptide of the sequence: Cys-pro-pro-pro-pro-Ser-Ser-Ile-Leu-Arg-Thr-Gln-Glu-Ser (amino acid residues 223-229 of influenza A virus Neuraminidase Protein NCBI sequence YP_009118627) (SEQ ID NO.:20) plus a N-terminal spacer, optionally coupled to a preferred immunogenic carrier, as described herein. Accordingly, this mimic represents an improved immunogen able to generate polyclonal antibodies against the preferred consensus amino terminal epitope sequences of the Influenza A and/B viral Neuraminidase protein.

Yet another embodiment of the invention is a preferred immunomimic that comprises an influenza neuraminidase peptide of the sequence: Cys-pro-pro-pro-pro-Ser-Thr-His-Ile-Glu-Glu-Ser-Ser-Ser-Tyr (amino acid residues 275-282 of influenza A virus Neuraminidase Protein NCBI sequence YP_009118627) (SEQ ID NO.:23) plus a N-terminal spacer, optionally coupled to a preferred immunogenic carrier, as described herein. Accordingly, this mimic represents an improved immunogen able to generate polyclonal antibodies against the preferred consensus amino terminal epitope sequences of the Influenza A and/B viral Neuraminidase protein.

Another embodiment of the invention is a preferred immunomimic that comprises an influenza neuraminidase peptide of the sequence: Cys-pro-pro-pro-pro-Ser-Thr-Ser-Arg-Asp-Asn-Trp-Lys-Gly-Ser-Asn-Arg-Pro (amino acid residues 292-302 of influenza A virus Neuraminidase Protein NCBI sequence YP_009118627) (SEQ ID NO.:25) plus a N-terminal spacer, optionally coupled to a preferred immunogenic carrier, as described herein. Accordingly, this mimic represents an improved immunogen able to generate polyclonal antibodies against the preferred consensus amino terminal epitope sequences of the Influenza A and/B viral Neuraminidase protein.

An additional embodiment of the invention is a preferred immunomimic that an influenza neuraminidase peptide of the sequence: Thr-Asp-Gly-Ser-Ala-Ser-Gly-Gln-Ala-Ser-Thr-Arg-Ile-Leu-Lys-Ser-Thr-pro-pro-pro-pro-Cys (amino acid residues 243-257 of influenza A virus Neuraminidase Protein NCBI sequence YP_009118627) (SEQ ID NO.:28) plus a C-terminal spacer, optionally coupled to a preferred immunogenic carrier, as described herein. Accordingly, this mimic represents an improved immunogen able to generate polyclonal antibodies against the preferred consensus amino terminal epitope sequences of the Influenza A and/B viral Neuraminidase protein.

Accordingly, this following embodiment is also a preferred immunomimic consisting of the influenza A nucleocapsid protein (NP) epitope sequences: Cys-pro-pro-pro-pro-Ser-Ser-Asp-Gln-Val-Arg-Glu-Ser-Arg-Asn-Pro-Gly-Asn-Ala-Glu-Ile-Glu-Asp-Leu-Ile (amino acid residues 240-257 of influenza A virus NP NCBI sequence ACZ46585.1) (SEQ ID NO.:29) plus a N-terminal spacer that contain one or more D-isomer amino acids, optionally coupled to a preferred immunogenic carrier, as described herein. Incorporation of spacers with specific peptides is a feature that can be important for proper positional presentation of adjacent immunogen peptides for imparting maximal targeted immunogenicity.

The following embodiment is another preferred immunomimic consisting of the influenza A Nucleocapsid protein (NP) epitope sequences: Phe-Leu-Ala-Arg-Ser-Ala-Leu-Ile-Leu-Arg-Gly-Ser-Val-Ala-His-Lys-Ser-Ser-Thr-pro-pro-pro-pro-Cys (amino acid residues 258-274 of influenza A virus NP NCBI sequence ACZ46585.1) (SEQ ID NO.:32) plus a C-terminal spacer that contain one or more D-isomer amino acids, optionally coupled to a preferred immunogenic carrier, as described herein. Incorporation of spacers with specific peptides is a feature that can be important for proper positional presentation of adjacent immunogen peptides for appropriate carrier conjugation.

The following embodiment is another preferred immunomimic consisting of the influenza A Polymerase basic protein-2 (PB2) epitope sequences: His-nLeu-Ala-Ile-Ile-Lys-Lys-nPhe-Thr-Ser-Gly-Arg-Gln-Glu-Lys-Asn-Pro-Ser-Leu-Ser-Thr-pro-pro-pro-pro-Cys (N-terminal amino acid residues 27-45 of influenza A virus PB2 NCBI sequence CAZ65595.1) (SEQ ID NO.:34) plus a C-terminal spacer that contain one or more D-isomer amino acids, optionally coupled to a preferred immunogenic carrier, as described herein. Incorporation of spacers with specific peptides is a feature that can be important for proper positional presentation of adjacent immunogen peptides for appropriate carrier conjugation; (where nLeu=L-Norleucine and nPhe=para-nitro-L-Phenylalanine).

The following embodiment is another preferred immunomimic consisting of the influenza A Polymerase basic protein-2 (PB2) epitope sequences: Cys-pro-pro-pro-pro-Ser-Thr-Arg-nLeu-Lys-Trp-nLeu-nLeu-Ala-nLeu-Lys-nPhe-Pro-Ile-Thr-Ala-Asp-Lys (amino acid residues 46-61 of influenza A virus PB2 NCBI sequence CAZ65595.1) (SEQ ID NO.:35) plus a N-terminal spacer that contain one or more D-isomer amino acids, optionally coupled to a preferred immunogenic carrier, as described herein. Incorporation of spacers with specific peptides is a feature that can be important for proper positional presentation of adjacent immunogen peptides for appropriate carrier conjugation; (where nLeu=L-Norleucine and nPhe=para-nitro-L-Phenylalanine).

The following embodiment is another preferred immunomimic consisting of the influenza A Polymerase basic protein-2 (PB2) epitope sequences: Val-Ala-nPhe-nLeu-Leu-Glu-Arg-Glu-Leu-Leu-Glu-Arg-Leu-Val-Arg-Lys-Thr-Arg-Phe-Leu-Pro-Ser-Thr-pro-pro-pro-pro-Cys (amino acid residues 203-219 of influenza A virus PB2 NCBI sequence CAZ65595.1) (SEQ ID NO.:38) plus a C-terminal spacer that contain one or more D-isomer amino acids, optionally coupled to a preferred immunogenic carrier, as described herein; (where nLeu=L-Norleucine and nPhe=para-nitro-L-Phenylalanine). Incorporation of spacers with specific peptides is a feature that can be important for proper positional presentation of adjacent immunogen peptides for appropriate carrier conjugation.

A following embodiment is another preferred immunomimic consisting of the influenza A Polymerase basic protein-2 (PB2) epitope sequences: Val-Ala-Gly-Gly-Thr-Ser-Ser-Val-nPhe-Ile-Glu-Val-Leu-His-Leu-Thr-Gln-Gly-Thr-Ser-pro-pro-pro-pro-Cys (amino acid residues 220-237 of influenza A virus PB2 NCBI sequence CAZ65595.1) (SEQ ID NO.:40) plus a C-terminal spacer that contain one or more D-isomer amino acids, optionally coupled to a preferred immunogenic carrier, as described herein. (where nPhe=para-nitro-L-Phenylalanine) Incorporation of spacers with specific peptides is a feature that can be important for proper positional presentation of adjacent immunogen peptides for appropriate carrier conjugation.

An ancillary embodiment is another preferred immunomimic consisting of the influenza A Polymerase basic protein-1 (PB1) epitope sequences: nPhe-Ile-Thr-Arg-Asn-Gln-Pro-Glu-Trp-Phe-Arg-Asn-Val-Leu-Ser-Ile-Ala-Pro-Ser-Thr-pro-pro-pro-pro-Cys (amino acid residues 324-342 of influenza A virus PB1 NCBI sequence ABD59820.1) (SEQ ID NO.:42) plus a C-terminal spacer that contain one or more D-isomer amino acids, optionally coupled to a preferred immunogenic carrier, as described herein. Incorporation of spacers with specific peptides is a feature that can be important for proper positional presentation of adjacent immunogen peptides for appropriate carrier conjugation.

The following embodiment is a preferred immunomimic consisting of the influenza A Polymerase basic protein-1 (PB1) epitope sequences: Ile-nLeu-Phe-Ser-Asn-Lys-nLeu-Ala-Arg-Leu-Gly-Lys-Gly-Tyr-nLeu-Phe-Glu-Ser-Lys-Ser-Thr-pro-pro-pro-pro-Cys (amino acid residues 343-360 of influenza A virus PB1 NCBI sequence ABD59820.1) (SEQ ID NO.:44) plus a C-terminal spacer that contain one or more D-isomer amino acids, optionally coupled to a preferred immunogenic carrier, as described herein. Incorporation of spacers with specific peptides is a feature that can be important for proper positional presentation of adjacent immunogen peptides for appropriate carrier conjugation.

An additional embodiment of the invention is a preferred immunomimic that comprises an influenza Matrix 1 pore mimetic peptide of the sequence: Cys-pro-pro-pro-pro-Thr-Thr-Asp-Leu-Glu-Ala-Leu-nLeu-Leu-Glu-Trp-Leu-Lys-Thr-Arg-Pro-Ile-Leu-Ser-Pro-Leu-Thr-Lys-Gly (amino acid residues 36-58 of influenza A virus M1 Protein ADB81462.1); plus a N-terminal spacer (SEQ ID NO.: 45) optionally coupled to a preferred immunogenic carrier, as described herein. Accordingly, this mimic represents an improved immunogen able to generate polyclonal antibodies against the preferred consensus epitope sequence of the influenza Matrix-1 pore protein. (where nL=L-Norleucine).

Also an additional embodiment of the invention is a preferred immunomimic that comprises an influenza Matrix 1 pore mimetic peptide of the sequence: Ile-Leu-Gly-Phe-Val-Phe-Tre-Leu-Thr-Val-Pro-Ser-Glu-Arg-Gly-Leu-Gln-Arg-Arg-Arg-nPhe-Thr-Ser-pro-pro-pro-pro-Cys (amino acid residues 59-79 of influenza A virus M1 Protein ADB81462.1); plus a C-terminal spacer (SEQ ID NO.: 48) optionally coupled to a preferred immunogenic carrier, as described herein. Accordingly, this mimic represents an improved immunogen able to generate polyclonal antibodies against the preferred consensus epitope sequence of the influenza Matrix-1 pore protein. (where nPhe=para-nitro-L-Phenylalanine).

In addition, another embodiment of the invention is a preferred immunomimic that comprises an influenza Matrix 1 pore mimetic peptide of the sequence: Cys-pro-pro-pro-pro-Thr-Thr-Leu-Gln-Ala-Tyr-Gln-Lys-Arg-nLeu-Gly-Val-Gln-nLeu-Arg (amino acid residues of influenza A virus M1 Protein ADB81462.1); plus a N-terminal spacer (SEQ ID NO.: 49) optionally coupled to a preferred immunogenic carrier, as described herein. Accordingly, this mimic represents an improved immunogen able to generate polyclonal antibodies against the preferred consensus C-terminal epitope sequence of the influenza Matrix-1 pore protein. (where nL=L-Norleucine).

An additional embodiment of the invention is a preferred immunomimic that comprises an influenza Matrix 2 pore mimetic peptide predicted by the sequence: Cys-pro-pro-pro-pro-Thr-Thr-Ile-Ile-Gly-Ile-Leu-His-Leu-Ile-Leu-Trp-Ile-Leu-Asp-Arg-Leu-Phe-Phe-Lys-Ser-Ile-nPhe-Arg-Leu-Phe (amino acid residues 32-55 of influenza A virus M2 Protein ADB59884.1); plus a N-terminal spacer (SEQ ID NO.: 51) optionally coupled to a preferred immunogenic carrier, as described herein. Accordingly, this mimic represents an improved immunogen able to generate polyclonal antibodies against the preferred consensus sequence of the influenza Matrix-2 pore protein. (where nPhe=para-nitro-L-Phenylalanine).

A further embodiment of this invention is another preferred immunomimic that comprises a mimetic T-Cell peptide of the influenza HA stalk sequence: Cys-po-pro-pro-pro-Thr-Thr-Ala-Ala-Asp-Leu-Lys-Ser-Tre-Gln-Glu-Ala-Ile-Asn-Gly (amino acid residues 378-390 of HA Protein P03452) plus a N-terminal 7 amino-acid spacer (SEQ ID NO.:53) optionally coupled to an immunogenic carrier, as described herein. Again, the non-toxic, more immunogenic Tetanus toxoid protein is a preferred immunogenic carrier in this embodiment.

In this context, the D-amino acid isomers can enable appropriate configuration for APC presentations, as well as enhance the persistence of the immunogen for APC presentation, yielding higher titers of antibody.

In conventional antibody technology, the induction of effective antibody responses by immunization with immune active conjugated carrier complexes, typically requires two or more administrations of immunogen, and it can take several weeks or months for the antibody titers to rise to the desired levels. By contrast, the improved immunogens of the present invention may induce effective levels of antibody shortly after the administration of initial course of immunogen. Levels of antibody thus elicited may stay elevated for several months and readily elevate to higher levels upon subsequent boosting by a single injection of immunogen.

In accordance with one aspect of the invention the novel anti-pan influenza immunogen that would be desired would generate polyclonal antibodies against all influenza viral HA protein species. Illustrative of such an immunogen is one that comprises (i) a selection of at least three or more mimitopes represented by SEQ ID NOs: 1-16, and 53-54 listed above optionally covalently coupled to (ii) an immunogenic carrier, suspended in a nano-emulsion and (iii) formulated using an aqueous and oily phase as described herein, respectively.

Similarly, another aspect of the invention the novel anti-pan influenza immunogen that would be desired would generate polyclonal antibodies against all influenza NA protein species. Illustrative of such an immunogen is one that comprises (i) a selection of at least two or more mimitopes represented by SEQ ID NO's: 19-28 listed above optionally covalently coupled to (ii) an immunogenic carrier, suspended in a nano-emulsion and (iii) formulated using an aqueous and oily phase as described herein, respectively.

In the present invention a preferred anti-pan influenza immunogen would be to generate polyclonal antibodies against the viral protein species using an immunogen that comprises (i) a selection of at least three or more mimitopes represented by SEQ ID NOs: 11, 12, 14 and 15 listed above optionally covalently coupled to (ii) an immunogenic carrier, such as the preferred recombinant carriers of DT, TT, drTeNT or HPV-VLP described above and suspended in the nano-emulsion and (iii) formulated using an aqueous and oily phase as described herein, respectively.

In the present invention a preferred anti-pan influenza viral immunogen would be to generate polyclonal antibodies against the viral protein species using an immunogen that comprises (i) a selection of at least two or more mimitopes represented by SEQ ID NO's: 24 and 25 listed above optionally covalently coupled to (ii) an immunogenic carrier, such as the preferred recombinant carriers of DT, TT, drTeNT or HPV-VLP or with alternative carrier mixtures described above and suspended in the nano-emulsion and (iii) formulated using an aqueous and oily phase as described herein, respectively.

Carriers/Vehicles Suitable for Use with the Immunogenic Compositions

In the present invention, the immunogenic carrier can be any suitable, high molecular-weight carrier, typically a protein or large (i.e., generally greater than 6000 kD) molecule of sufficient molecular complexity that can elicit an immune response towards a haptene or peptide sequence that is covalently linked to it. The category of suitable immunogenic carriers is exemplified by but not limited to the non-toxic toxoidal proteins like diphtheria toxoid (DT), tetanus toxoid (TT), pertussin toxoid (PT), or recombinant toxoids like PrimeBio Inc., novel, non-toxic but immunogenic recombinant Tetanus (drTeNT) protein. Among these, diphtheria toxoid (DT) or tetanus toxoid (TT) are preferred immunogenic carriers. This category can also encompass particulate carriers such as the nanoparticulate calcium phosphate (nCAP) described by Qing He et al., Clin. Diag. Lab Immunology 7:899-903 (2000).

A suitably acceptable vehicle or carrier denotes a medically safe, non-toxic substance that will convey an immunogen without diminishing its immunogenic effect. A suitable vehicle therefore, can be a liquid emulsion, as further described below, or it can be a stable particulate substance, e.g., as a pharmaceutically safe lyophilized powder or pharmaceutically acceptable hydrocolloidal gel or recombinant, synthesized, non-infectious virus like particles (VLP) that are now FDA approved in commercial vaccines. (FIELDS VIROLOGY, 6^{th} ed. Vol. I, D. M. Knipe & P. Howley (eds.), Lippincott Williams & Wilkins (2013)).

In an embodiment, a preferred form of pharmaceutically acceptable vehicle or carrier is an emulsion of an aqueous phase, containing the polypeptide immunogen, and an oily phase. The oily phase comprises at least one biodegradable oil, immiscible with the aqueous phase, that is non-toxic in the dosage range of intended administration. The oil can be natural or synthetic, and there are many such oils available, which are generally recognized as safe and meet international regulatory acceptance for therapeutic vaccine use. Illustrative of such suitable oils are squalene, squalane, Sorbitan monooleate, Polysorbate 40, and Polysorbate 80. A preferred oily phase comprises four or five of these component oils in the oily phase.

The emulsions are mixed for a sufficient amount of time to generate nanoparticles. The preferable size of a majority (i.e., more than about 50%) of the nanoparticles in the emulsion is 250 nanometers or less. For example, squalene/squalane-based emulsions can be mixed in an ice water bath at kept at (0-4° C.) low temperatures, using an IKA overhead stirrer suitable for 8,000 rpm mixing and/or Microfluidics M-110P Microfluidizer™ mixer or equivalent, for a time period suitable for generating nano-sized emulsification particles.

In addition, the oily phase may contain one or more separate emulsifiers, such as alpha-tocopherol, aluminum monostearate or an adjuvant-active saccharide oleate or saccharide stearate ester.

Adjuvants Suitable for Use with the Immunogenic Compositions

In accordance with another aspect of the invention, either the oily or aqueous phase of an emulsion as described above contains at least one adjuvant that is distinct from the immunogenic carrier component of the polypeptide immunogen. There is a wide range of known adjuvants, any one or more which may be considered for use in this invention.

Non-limiting exemplary adjuvants include: cyclic-di-purine mononucleotides, cyclic diguanylate, or synthetic STING activators, as well as toll-like receptor agonists Imiquimod, cyclic diadenylate, Isoprinosine, trehalose dimycolate, QS-21, alpha-galactosylceramide (C-GalCer), and alpha-glucosylceramide (C-GluCer).

For this adjuvant role, moreover, the present invention comprehends the use of a material that, if not typically deemed an adjuvant per se, is immunostimulatory nevertheless. Non-limiting exemplary materials include Ergamisol, Cimetidine, Praziquantel, uric acid, mannan and derivatives of mannan, and vitamins A, D3 and vitamin E.

### Exemplary Method of Making the Disclosed Immunogenic Compositions

In accordance with a preferred aspect of the invention, the peptides disclosed herein can be conjugated to amino groups present on the tetanus toxoid (TT) immunogenic carrier. The linkage was via the terminal peptide cysteine residue, utilizing hetero-bifunctional linking agents containing typically a succinimidyl ester at one end and maleimide at the other end of the linking agent. To accomplish the linkage between the peptides described herein and the carrier, the cysteine of the peptide was first reduced. The dry peptide was dissolved in 0.1M sodium phosphate buffer, pH 7-9, with a 5-50 molar excess of dithiothreitol. The peptide was lyophilized and stored under vacuum until used. Typically, the carrier protein is activated by treatment with the hetero-bifunctional linking agent epsilon-maleimidocaproic acid N-hydroxysuccinimide ester (EMCS), in proportions sufficient to achieve activation of approximately 25 free amino groups per 10⁵ molecular weight of carrier.

Preparation of Purified Tetanus Toxoid: Typically, if TT is used it was purified by ultrafiltration. Final concentration of recovered purified TT was expected to be 5-40 mg/ml. The purity was determined by chromatography (SEC HPLC), protein concentration (Bradford protein assay or Lowry assay), and free amino-groups (by ninhydrin). Peptides were obtained commercially (Biosyn Corp, USA), and reduced peptide with known purity and content was used for conjugation. Peptides were reduced with tris (2-carboxyethyl)-phosphine-HCl (TCEP), and the mixture was used in the conjugation. Ellman's assay can be used to determine free sulfhydryl groups.

Conjugation of Peptide-TT. After calculating the quantity of peptide to react with the maleimido-TT, the peptide was added to the M-TT solution. The peptide-TT conjugate was purified by ultrafiltration filtered. The conjugates of the peptides to be linked to carrier via EMCS and are to be separated from other components of the mixture by low pressure chromatography at 4° C. over a G50 Sephadex column equilibrated with 0.1-0.5M ammonium bicarbonate. In each case the conjugate was eluted in the column Void volume and was lyophilized and stored, desiccated, at 4-0° C. until use.

The conjugate may be characterized as to immunomimic peptide content by a number of methods known to those skilled in the art including weight gain, amino acid analysis, etc. Conjugates of peptides to carrier proteins produced by these methods are determined by amino acid analysis to have 10-30 moles of peptide per 10⁴-10⁶ MW of carrier and all are considered suitable as immunogens for immunization of test animals.

### Methods of Administering the Compositions

In a preferred embodiment, the vaccine compositions of the invention are administered in two doses, e.g., an initial dose followed by a second booster dose. For example, the second dose of the vaccine composition can be administered at least 2 weeks after the first dose, preferably is administered 3 weeks after the first dose, and more preferably is administered 4 weeks after the first dose. The amount of the disclosed peptides can be the same in the first dose and the second dose, or the amounts can be different. For example, the initial dosage can comprise about 25 to about 500 micrograms (μg) peptides (e.g., 25, 50 or 100 μg) followed in two to four weeks by a secondary dose of the same or double dosage as defined in clinical trial phase-1 dosage escalation studies.

The vaccine compositions can be administered by any desired route, e.g., subcutaneous, intramuscular, or mucosal (e.g., intranasal or sublingual) administration. The vaccine compositions can be formulated to be used in such administration methods.

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Example 1. Preparation of Peptides

Peptides were prepared by standard solid-state synthesis commercial methods. Each peptide was characterized as to amino acid content and purity. Peptides with the amino acid sequences listed above SEQ ID: 1-54, were thus synthesized. In these sequences, as in others of the present description, an amino acid beginning in a capital letter is an L-isomer amino acid, while at least one or more in a lower-case letter is a D-isomer.

In accordance with a preferred aspect of the invention, each of these peptides was conjugated to amino groups present on a toxoidal (e.g., DT, or TT) or recombinant (e.g., drTeNT) or VLP immunogenic carrier. The linkage was via the terminal peptide cysteine residue, utilizing heterobifunctional linking agents containing typically a succinimidyl ester at one end and maleimide at the other end of the linking agent. To accomplish the linkage between either of the Mimic Peptides listed above and the carrier, the cysteine of the peptide was first reduced. The dry peptide was dissolved in 0.1M sodium phosphate buffer, pH 7-9, with a 5-50 molar excess of dithiothreitol. The peptide was lyophilized and stored under vacuum until used. Typically, the carrier protein is activated by treatment with the hetero-bifunctional linking agent such as an epsilon-maleimidocaproic acid N-hydroxysuccinimide ester (e.g., EMCS), in proportions sufficient to achieve activation of approximately 25 free amino groups per 10⁵ molecular weight of carrier.

Preparation of Purified Diphtheria or Tetanus Toxoid: Typically, if DT or TT is used, it was purified by ultrafiltration. Final concentration of recovered purified DT/TT was expected to be 5-40 mg/ml. The purity was determined by chromatography (SEC HPLC), protein concentration (Bradford protein assay or Lowry assay), and free amino-groups (by ninhydrin). Peptides were obtained commercially (Biosyn Corp, USA), and reduced peptide with known purity and content was used for conjugation. Peptides were reduced with tris (2-carboxyethyl)-phosphine-HCl (TCEP), and the mixture was used in the conjugation. Ellman's assay can be used to determine free sulfhydryl groups.

Conjugation of Peptide-DT/TT. After calculating the quantity of peptide to react with the maleimido-DT/TT, (mDT/mTT) the peptide was added to the m-DT/mTT solution.

The peptide-DT/TT conjugate (cDT/cTT) was purified by ultrafiltration filtered. The conjugates of the peptides to be linked to carrier via conjugate linker such as EMCS and are to be separated from other components of the mixture by low pressure chromatography at 4° C. over a G50 Sephadex column equilibrated with 0.1-0.5M ammonium bicarbonate. In each case the conjugate was eluted in the column Void volume and was lyophilized and stored, desiccated, at 4-0° C. until use.

The conjugate may be characterized as to immunomimic peptide content by a number of methods known to those skilled in the art including weight gain, amino acid analysis, etc. Conjugates of peptides to carrier proteins produced by these methods are determined by amino acid analysis to have 10-30 moles of peptide per 10⁴-10⁶ MW of carrier and all are considered suitable as immunogens for immunization of test animals.

### Example 2. Characterization of the Immunogenic Peptides

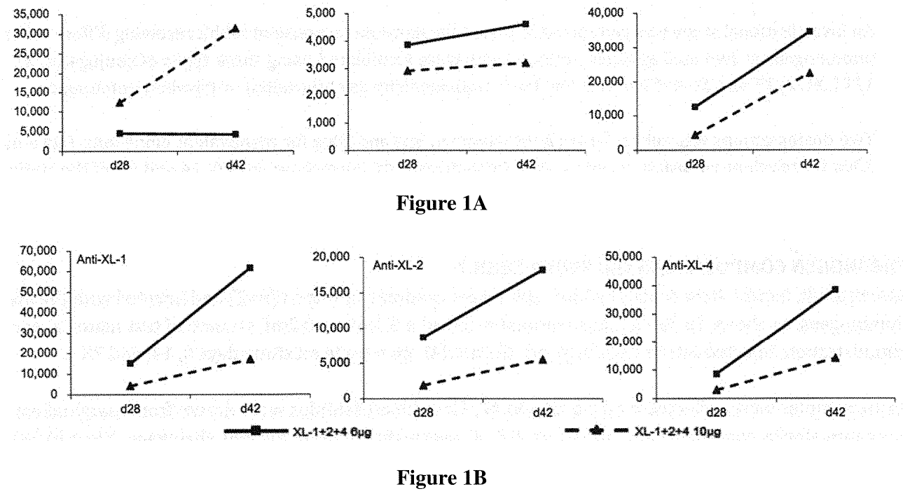

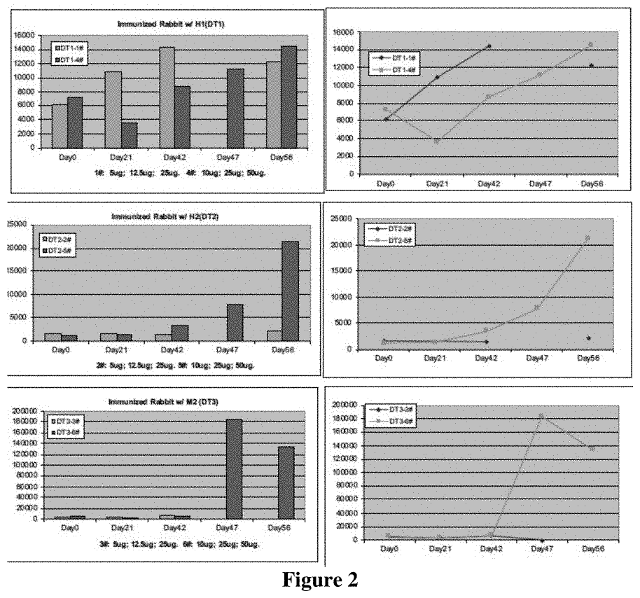

The titers of anti-SEQ ID No.:1 mimitope (XL-1) conjugated with TT; anti-SEQ ID No.:17 mimitope (XL-2) conjugated with TT, and anti-SEQ ID No.:16 mimitope (XL-4) conjugated with TT were measured in each animal sera sample by standard ELISA method (ELISA: Methods and Protocols (Methods in Molecular Biology (1318)) 2015th Edition, R. Hnasko Ed.) Sixteen adult female Rabbits (New Zealand whites) were randomly grouped (n=2) and received different monovalent immunogens with two dose levels of 5 μg and 10 μg in 0.1 ml and 0.2 ml emulsion, respectively. Immunogens were injected intramuscularly into the hind thigh at days 0, 14 and 28; samples of sera were collected on days 28 and 42 for immunogenicity (ELISA) assays. All titers were expressed relative to the pre-bleed control sera (negative sera). The Individual mean antibody (Ab) titer for each group is presented in Tables I and 2 below and FIGS. 1A-1B and FIG. 2.

TABLE 1
Immunogen Compositions and Study Groups. Sequences
(L-1, L-2, L-4) are as listed in FIG 2.

| Groups (n = 2) | Conjugates | Dosage |
|---|---|---|
| 1) L-1 Low | XL-1/TT | 5 μg/0.1 mL × 3 |
| 2) L-1 High | XL-1/TT | 10 μg/0.2 mL × 3 |
| 1) L-2 Low | XL-2/TT | 5 μg/0.1 mL × 3 |
| 2) L-2 High | XL-2/TT | 10 μg/0.2 mL × 3 |
| 1) L-3 Low | XL-4/TT | 5 μg/0.1 mL × 3 |
| 2) L-3 High | XL-4/TT | 10 μg/0.2 mL × 3 |
| 1) L-4m × Low | XL-1 + 2 + 4/TT | 6 μg/0.1 mL × 3 |
| 2) L-4m × High | XL-1 + 2 + 4/TT | 10 μg/0.1 mL × 3 |

Note that XL-1 corresponds to SEQ ID No.:1, which is an HA-1 epitope conjugated to tetanus toxin; XL-2 corresponds to SEQ ID No.:17, which is an M2 pore epitope conjugated to tetanus toxin; and XL-4 corresponds to SEQ ID No.:16, which is an HA-2 epitope conjugated to tetanus toxin.

TABLE 2
Actual mean titers in rabbits following three injections
(Low & High) of immunogens: XL-1; XL-2; XL-4; and mixtures
of all three (Low & High) as shown in FIG. 1A-1B.
Group# 1

| | | Anti-XL-1 | |
|---|---|---|---|
| Immunogen/Dosage | | D 28 | D 42 |
| XL-1 Low | #1 | 8,229 | dead |
| 5 μg/0.1 ml | #2 | 860 | 4,249 |
| 20140701-1 | Mean | 4,544 | 4,249 |

Group# 2

| | | Anti-XL-1 | |
|---|---|---|---|
| Immunogen/Dosage | | D 28 | D 42 |
| XL-1 High | #1 | 13,157 | 30,804 |
| 10 μg/0.2 ml | #2 | 11,505 | 31,886 |
| 20140701-1 | Mean | 12,331 | 31,345 |

Group# 3

| | | Anti-XL-2 | |
|---|---|---|---|
| Immunogen/Dosage | | D 28 | D 42 |
| XL-2 Low | #1 | 1,687 | 1,787 |
| 5 μg/0.1 ml | #2 | 6,029 | 7,429 |
| 20140701-2 | Mean | 3,858 | 4,608 |

Group# 4

| | | Anti-XL-2 | |
|---|---|---|---|
| Immunogen/Dosage | | D 28 | D 42 |
| XL-2 High | #1 | 5,391 | 4,556 |
| 10 μg/0.2 ml | #2 | 437 | 1,858 |
| 20140701-2 | Mean | 2,914 | 3,207 |

Group# 5

| | | Anti-XL-4 | |
|---|---|---|---|
| Immunogen/Dosage | | D 28 | D 42 |
| XL-4 Low | #1 | 4,071 | 14,844 |
| 5 μg/0.1 ml | #2 | 20,907 | 54,495 |
| 20140701-3 | Mean | 12,489 | 34,670 |

Group# 6

| | | Anti-XL-4 | |
|---|---|---|---|
| Immunogen/Dosage | | D 28 | D 42 |
| XL-4 High | #1 | 4,244 | 8,450 |
| 10 μg/0.2 ml | #2 | 4,528 | 36,796 |
| 20140701-3 | Mean | 4,386 | 22,623 |

Group# 7

| | | Anti-XL-1 | | Anti-XL-2 | | Anti-XL-4 | |
|---|---|---|---|---|---|---|---|
| Immunogen/Dosage | | D 28 | D 42 | D 28 | D 42 | D 28 | D 42 |
| XL-1 + 2 + 4 Low | #1 | 15,006 | 61,602 | 8,691 | 18,221 | 8,647 | 38,597 |
| 5 μg/0.1 ml | #2 | dead | dead | dead | dead | dead | dead |
| 20140701-4/1 | Mean | 15,006 | 61,602 | 8,691 | 18,221 | 8,647 | 38,597 |

Group# 8

| | | Anti-XL-1 | | Anti-XL-2 | | Anti-XL-4 | |
|---|---|---|---|---|---|---|---|
| Immunogen/Dosage | | D 28 | D 42 | D 28 | D 42 | D 28 | D 42 |
| XL-1 + 2 + 4 High | #1 | 4,740 | 14,147 | 2,309 | 3,642 | 2,939 | 10,858 |
| 10 μg/0.1 ml | #2 | 3,594 | 19,657 | 1,416 | 7,406 | 3,033 | 17,474 |
| 20140701-4/2 | Mean | 4,167 | 16,902 | 1,863 | 5,524 | 2,986 | 14,166 |

### Example 3. In Vivo Efficacy of the Immunogenic Peptides

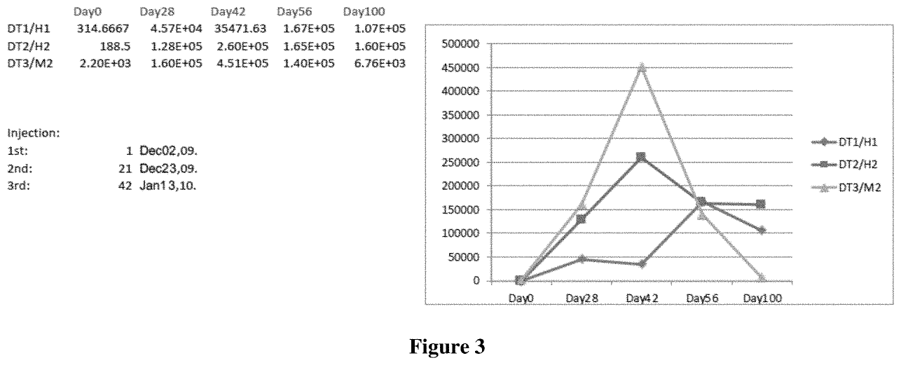

To determine in vivo the efficacy of the mimetic immunogens in generating anti-epitope antibodies against influenza epitope targets in order to later test their ability to protecting mice from lethal doses of influenza viruses, the inventors immunized flu susceptible mice raised in the BL3 laboratories of the College of Veterinary Medicine, China Agricultural University, 100193 Beijing, China; (work supervised by Dr. Wang Bin) with three initial doses (2.5 μg) of SEQ ID No.:1 mimitope (HA1) conjugated with DT (immunogen DT1), and SEQ ID No.: 16 mimitope (HA2) conjugated with DT (immunogen DT2) and SEQ ID No.:17 mimitope (M2) conjugated with DT (immunogen DT3 Mice sera from each animal was drawn as depicted and titered by standard ELISA method (ELISA: Methods and Protocols (Methods in Molecular Biology (1318)) 2015th Edition, R. Hnasko Ed.). Six adult female mice (Balb/c) were randomly grouped (n=6) and received different monovalent immunogens with three doses of 2.5 μg in 0.1 ml of a proprietary emulsion. Immunogen was injected intraperitoneally at days 0, 21 and 42; samples of sera were collected on days 1, 28, 42, 56, and 100 for immunogenicity (ELISA) assays. All titers were expressed relative to the pooled pre-bleed control sera (negative sera). The individual mean antibody (Ab) titer kinetics for the low dose (2.5 μg) group is presented in FIG. 3.

The peak titers of DT2/HA2 and DT3/M2 was at day 42; the peak titer for DT1/HA1 occurred at day 56. Average titers were highest for DT3/M2 immunogen.

### Example 4. In Vivo Efficacy of the Immunogenic Peptides

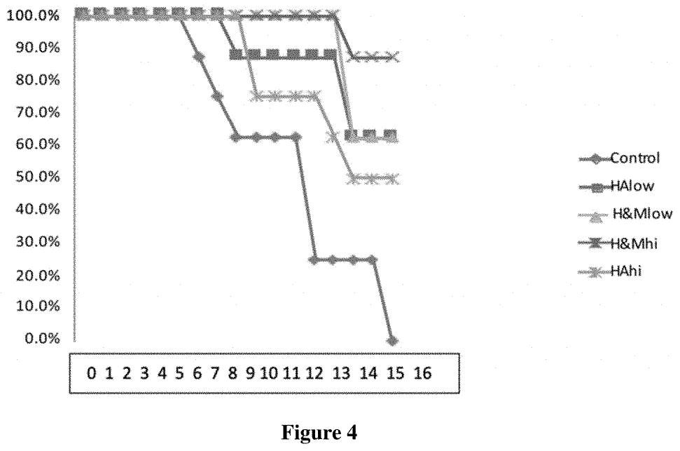

To determine the in vivo efficacy of the mimetic immunogens to protect mice from lethal doses (5× the lethal LD₅₀ dose of the bird H5N1 strain) of influenza viruses, the inventors immunized flu susceptible mice raised in the BL3 laboratories of the College of Veterinary Medicine, China Agricultural University, 100193 Beijing, China; (work supervised by Dr. Wang Bin) with three initial doses (of low (1.25 μg) and high (5.0% g) dosages) of SEQ ID No.:1 mimitope (HA1) conjugated with DT (immunogens HA_{low}; HA_{high}), and with a mix of SEQ ID No.:17 mimitope (M2) conjugated with DT (as immunogens HA_{low}+M_{low}; HA_{high}+M_{high}). Mice sera from each animal was drawn as depicted and titered by standard ELISA method (ELISA: Methods and Protocols (Methods in Molecular Biology (1318)) 2015th Edition, R. Hnasko Ed.). Six adult female mice (Balb/c) were randomly grouped (n=5) and received the different low and high dosages of monovalent and mixture of immunogens at the specified two dose levels of 1.25 μg and 5.0 μg in 0.1 ml and 0.2 ml emulsion, respectively, including the control group which received only 0.2 ml emulsion minus immunogen. All immunogens were injected intraperitoneally at days 0, 14 and 28; on day 38 samples of 5×LD₅₀H5N1 was introduced intranasally into the indicated immunized groups (5/group) of mice. The individual groups were monitored for activity and viability for 16 days, when all of the control mice had perished. Survival is presented in Table 3 below and in FIG. 4.

TABLE 3
(Balb/c Mice Survival following three initial administrations of immunogens
HA-1 and the mix (HA-1 + M2 pore) at low and high dosages; All mice
were compared to Controls after exposure at day 38 with 5X LD50 H5N1).

| | Day 0 | Day 1 | Day 2 | Day 3 | Day 4 | Day 5 | Day 6 | Day 7 | Day 8 |
|---|---|---|---|---|---|---|---|---|---|
| control | 100.0% | 100.0% | 100.0% | 100.0% | 100.0% | 100.0% | 87.5% | 75.0% | 62.5% |
| HA_{low} | 100.0% | 100.0% | 100.0% | 100.0% | 100.0% | 100.0% | 100.0% | 100.0% | 87.5% |
| H&1516 | 100.0% | 100.0% | 100.0% | 100.0% | 100.0% | 100.0% | 100.0% | 100.0% | 100.0% |
| H&M_{high} | 100.0% | 100.0% | 100.0% | 100.0% | 100.0% | 100.0% | 100.0% | 100.0% | 100.0% |
| HA_{high} | 100.0% | 100.0% | 100.0% | 100.0% | 100.0% | 100.0% | 100.0% | 100.0% | 100.0% |

| | Day 9 | Day 10 | Day 11 | Day 12 | Day 13 | Day 14 | Day 15 | Day 16 |
|---|---|---|---|---|---|---|---|---|
| control | 62.5% | 62.5% | 62.5% | 25.0% | 25.0% | 25.0% | 25.0% | 0% |
| HA_{low} | 87.5% | 87.5% | 87.5% | 87.5% | 87.5% | 62.5% | 62.5% | 62.5% |
| H&1516 | 100.0% | 100.0% | 100.0% | 100.0% | 100.0% | 62.5% | 62.5% | 62.5% |
| H&M_{high} | 100.0% | 100.0% | 100.0% | 100.0% | 100.0% | 87.5% | 87.5% | 87.5% |
| HA_{high} | 75.0% | 75.0% | 75.0% | 75.0% | 62.5% | 50.0% | 50.0% | 50.0% |

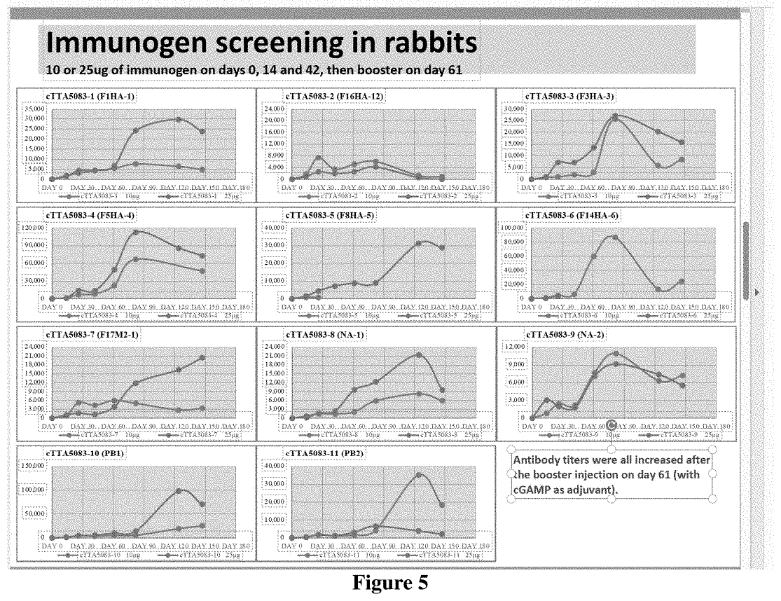

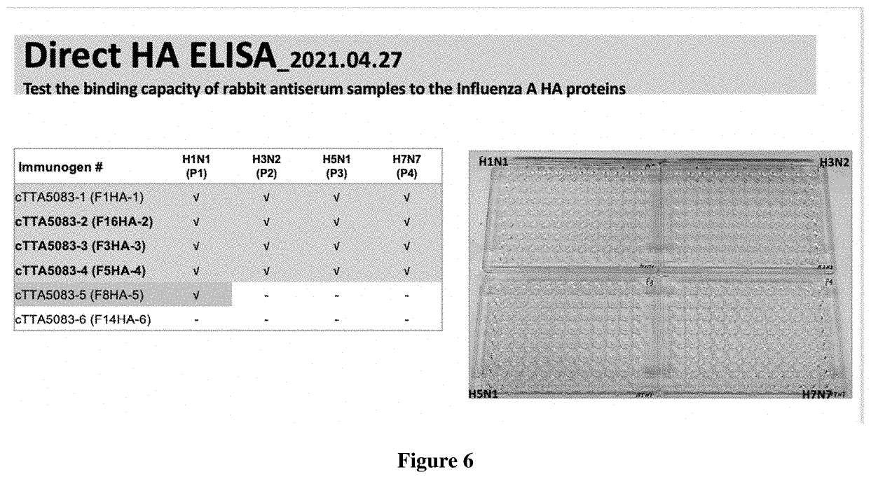

The inventors then determined if the immunogens were capable of binding to and possibly neutralizing hemagglutinin (HA) peptides of various viruses (FIG. 5). Titers of eleven TT-conjugated immunogens in rabbits were determined after an initial series (days 0, 14, 21) of two doses (10 mcg/dose & 25 mcg/dose) followed by a booster dose on day 61 with cGAMP as an adjuvant. Antisera were harvested at day 143 for HA-ELISA studies (shown in FIG. 6) on avian, swine, equine and recombinant human influenza HA viral protein binding studies. Antibody titers of all eleven immunogenic peptides were increased after the booster dose, using cGAMP as an adjuvant.

For succeeding studies, the rabbit antisera obtained from conjugated Tetanus toxoidal (cTT) peptides A5083-1 to A5083-6 representing sequence IDs: A5083-1=HA Seq ID No.: 1; A5083-2=HA Seq ID No.: 16; A5083-3=HA Seq ID No.: 3; A5083-4=HA Seq ID No.: 5; A5083-5=HA Seq ID No.: 8, and A5083-6=HA Seq ID No.: 14.

Each of these rabbit antisera was tested by ELISA for binding to purified recombinant synthesized his-tagged HA peptides (rHA-peptides: P1; H1N1 human flu HA; P2 H3N2 swine flu HA; P3 H5N1 avian flu HA; and P4 H7N7 equine flu HA) obtained from SinoBiologicals, PRC affixed to nickel coated microwell plates (Thermo Scientific Pierce Nickel Coated White 96-well plates) as follows. The four flu HA peptides were coated at a concentration of 22 ng per well and allowed to incubate overnight at 2-8° C. The plates were then blocked with 200 ul of 4% milk per well at room temperature for 2 hours. Next, 100 ul of rabbit antisera against influenza antigens (post-day 147) were added to each well and incubated at room temperature for 2 hours. Each sample was tested for four dilutions and performed in duplicate. Next, 100 ul of the second antibody, an AP-conjugated goat anti-rabbit IgG antibody, was added to each well and incubated at room temperature for 2 hours. Finally, 100 ul of 1 mg/ml pNPP substrate was added to each well, allowed to react at room temperature for 10 minutes, and the reaction was stopped with 1.0 M NaOH. The plates were then read by plate reader.

Negative controls were negative/positive control was SinoBiological's monoclonal (Mab) against H1N1 HA epitope (only).

The ELISA data (FIG. 6) showed that immunogens against peptides A5083-1 to 5083-4 elicited antisera that bound to and recognized flu recombinant HA proteins of human, swine, avian and equine type A influenzas, strongly implying broad recognition.

### Example 5. Administration of the Vaccine Compositions

A vaccine composition as described herein can be administered in two doses. Preferably, the first dose is administered, and the second dose is administered at least two weeks after the first dose, at least three weeks after the first dose, or at least four weeks after the first dose. The amount of the disclosed peptides can be the same in the first dose and the second dose, or the amounts can be different. For example, the initial dosage can comprise about 25 to about 500 micrograms (μg) peptides (e.g., 25, 50 or 100 μg) followed in two to four weeks by a secondary dose of the same or double dosage as defined in clinical trial phase-1 dosage escalation studies.

The vaccine compositions can be administered by any suitable method, e.g., subcutaneously, intramuscularly, or mucosally (e.g., intranasal or sublingual).

### List of Items

1. A polypeptide immunogenic composition comprising:
   (A) at least one mimetic peptide comprising: (i) at least one consecutive amino acid sequence of conserved type A & B influenza viruses or a processed subspecies thereof, selected from the HA1 and/or HA2 protein domain groups and/or Neuraminidase (n1-N10) protein domain and/or Nucleocapsid protein domains and/or the M1 and/or M2 pore protein domain and/or the basic Polymerases B1 and/or B2 protein domains of the influenza viruses synthesized covalently to (ii) a 7 amino-acid spacer moiety, and conjugated to (iii) an immunogenic carrier coupled to said mimetic peptide(s),
   wherein the mimetic peptide(s) comprises a chemically synthesized and/or modified amino acid and/or amino acid epitope that augments the immunogenicity of the polypeptide immunogen, and
   wherein the amino acid sequence is from about 6 amino acid residues to about 30 amino acids, comprising a mixture of L- and D-enantiomeric amino acids.
2. The polypeptide immunogenic composition according to item 1, further comprising
   at least 3 to 5 oligopeptides comprising amino acid sequences and/or mimetic sequences selected from the group consisting of SEQ ID NOs: 1-16 and amino acid sequences and/or mimetic sequences selected from the group consisting of SEQ ID NOs: 17-54; and
   a pharmaceutically acceptable carrier.
3. The polypeptide immunogenic composition according to item 1 or 2, wherein the at least one mimetic peptide comprises:
   at least one oligopeptide having an overall length of from 6-50 amino acids, optionally from 6-30 amino acids, selected from the group consisting of SEQ ID NOs:1-16 and amino acid sequences and/or mimetic sequences selected from the groups consisting of SEQ ID NOs: 17-54 and a pharmaceutically acceptable carrier in a nano-sized emulsified preparation suitable for parenteral or mucosal application(s).
4. The polypeptide immunogenic composition according to any of items 1-3, wherein at least the mimetic peptide(s) administered include a mixture of defined peptides selected from the group consisting of SEQ ID No.:1 to SEQ ID: NO.: 54.
5. The polypeptide immunogenic composition according to any of items 1-4, wherein the immunogenic carrier is selected from the group consisting of toxoidal proteins like diphtheria toxoid (DT), tetanus toxoid (TT), pertussin toxoid (PT), or recombinant toxoidal-like protein, a non-toxic but immunogenic recombinant Tetanus (non-toxoidal) protein, and particulate carriers such as the nanoparticulate calcium phosphate (nCAP) carrier substance, with or without ancillary protein carriers.
6. The polypeptide immunogenic composition according to any of items 1-5, wherein the immunogenic carrier is selected from commercially available FDA approved toxoids such as tetanus/diphtheria toxoid or a recombinant non-toxoid tetanus or any commercially available FDA approved virus like particle (VLP).
7. An immunogenic composition comprising a therapeutically effective amount of the polypeptide immunogenic composition according to any of items 1-6, and a pharmaceutically acceptable carrier.
8. The immunogenic composition according to item 7, wherein the pharmaceutically acceptable carrier comprises an emulsion of an aqueous phase, in which the at least one mimetic peptide is selected from the group consisting of SEQ ID NOs: 1-54 and is dissolved or in suspension, and
   an oily phase,
   wherein such aqueous emulsion comprises from 25% to 50% by weight of the composition.
9. The immunogenic composition according to items 7 or 8, wherein said oily phase comprises at least one or more of squalene, squalane, sorbitan monooleate, Polysorbate 40, Poly Sorbate 80, and polysorbates.
10. The immunogenic composition according to any of items 7-9, wherein said oily phase comprises at least one emulsifier agent capable of enabling oil/water (o/w) or water/oil (w/o) mixtures to form nano-sized emulsions (<500 nanometers); stable at 2-6° C. for at least one year or more.
11. The immunogenic composition according to any of items 7-10, wherein either said oily phase or said aqueous phase contains at least one adjuvant.
12. The immunogenic composition according to item 11, wherein said adjuvant is selected from the group consisting of: cyclic-di-purine mononucleotides, cyclic diguanylate, Imiquimod, cyclic diadenylate, Isoprinosine, trehalose dimycolate, QS-21, alpha-galactosylceramide (C-GalCer), Polyinosinic-polycytidylic acid (poly I:C), non-methylated CpG oligodeoxynucleotides (ODN), alpha-glucosylceramide (C-GluCer), and combinations thereof.
13. The immunogenic composition according to items 12 or 13, wherein said adjuvant is selected from the group consisting of Ergamisol, Cimetidine, Praziquantel, uric acid, mannan and derivatives of mannan, one or more of the vitamins A, D3 and vitamin E, and combinations thereof.
14. The composition of any preceding item wherein said w/o or o/w emulsion for the immunogen is used in conjunction with a pharmaceutically approved protein solubilizer.
15. The composition of any preceding item wherein said protein solubilizer is urea, DMSO or glycerin.
16. A immunization method comprising administering to a patient having any influenza infection, an immunogenic composition according to any preceding item such that an antibody response is elicited in said patient, optionally wherein the immunogenic composition is administered in more than one dose.
17. The compositions and methods of any preceding item, wherein the immunogenic composition elicits the production of antibodies.
18. The compositions and methods of any preceding items, wherein the immunogenic composition elicits an adaptive immune response, humoral immune response or an innate immune response.
19. The composition of any of preceding items, wherein the immune response is a CD8+ and/or CD4⁺ T-cell response, including a Th-1, Th-2 and Th-17 response.
20. A method for treating or reducing the likelihood of a disease in a human subject, the method comprising:
   administering an immunogenic composition of any preceding items to a human subject in need thereof, optionally wherein the immunogenic composition is administered in more than one dose.
21. A method for generating an immune response to two or more influenza hemagglutinin subtypes and/or influenza types A and B encoded proteins, comprising:
   administering to a subject an effective amount of the immunogenic compositions of any preceding item, thereby generating the immune response, optionally wherein the immunogenic composition is administered in more than one dose.
22. A vaccine composition comprising at least one peptide comprising an amino acid sequence selected from the group consisting of SEQ ID NOs: 1-54, optionally coupled to a spacer peptide comprising two hydroxy amino acids coupled to either SEQ ID NO: 55 or 56.
23. The vaccine composition of item 22, wherein the at least one peptide is further coupled to an immunogenic carrier.
24. The vaccine composition of items 22 or 23, further comprising an adjuvant, excipient, and/or pharmaceutically acceptable carrier.
25. The vaccine composition of any of items 22-24, wherein the vaccine composition is formulated for intramuscular, subcutaneous, or mucosal administration.
26. The vaccine composition of any of items 22-25, wherein the vaccine composition comprises at least three peptides comprising an amino acid sequence selected from the group consisting of SEQ ID NOs: 1-54.
27. The vaccine composition of any of items 22-25, wherein the vaccine composition comprises at least four peptides comprising an amino acid sequence selected from the group consisting of SEQ ID NOs: 1-54.
28. The vaccine composition of any of items 22-25, wherein the vaccine composition comprises at least five peptides comprising an amino acid sequence selected from the group consisting of SEQ ID NOs: 1-54.
29. The vaccine composition of any of items 22-28, wherein the vaccine composition comprises one or more peptides comprising an amino acid sequence selected from the group consisting of (i)(a) SEQ ID NOs: 1, 3, 5, 10, 12, 14, 16, 17, 19, 23, 25, 28, 29, 32, 34, 35, 40, 44, 45, 47, 49, 51, and 53; and/or (i)(b) SEQ ID NOs: 2, 4, 6, 8, 9, 11, 13, 15, 18, 20, 21, 24, 26, 27, 30, 31, 33, 36, 37, 38, 39, 42, 46, 50 and 54 and/or (i)(c) SEQ ID NOs: 1, 3, 5, 8, 10, 12, 14, 15, 17, 19, 23, 25, 27, 29, 31, 34, 37, 42, 44, 46, 47, 48, 51, and 53.
30. The vaccine composition of any of items 22-28, wherein the vaccine composition comprises one or more peptides comprising an amino acid sequence selected from the group consisting of SEQ ID NOs: 1-16, and (i) 17-28, and/or (ii) 18-19, and/or (iii) 20-28, and/or (iv) 33-40 and/or (v) 41-44 and/or (vi) 53-54.
31. The vaccine composition of any of items 22-28, wherein the vaccine composition comprises one or more peptides comprising an amino acid sequence selected from the group consisting of SEQ ID NOs: 19-28 and/or 33-44.
32. The vaccine composition of any of items 22-28, wherein the vaccine composition comprises one or more peptides comprising an amino acid sequence selected from the group consisting of SEQ ID NOs: 10, 11, 12, 14, and 15.
33. The vaccine composition of any of items 22-28, wherein the vaccine composition comprises one or more peptides comprising an amino acid sequence selected from the group consisting of SEQ ID NOs: 1-16 and/or 23, 24 and 25.
34. The vaccine composition of any of items 22-28, wherein the vaccine composition comprises one or more peptides comprising an amino acid selected from the group consisting of (a) SEQ ID NOs: 10, 11, 12, 14, 15, 17, 23, 25, and/or 27; (b) SEQ ID NOs: 15 and 17-28; and/or (c) SEQ ID NOs: 17, 23, 25, and 27.
35. The vaccine composition of any of items 22-34, wherein the vaccine composition comprises:
   at least one peptide comprising an amino acid sequence selected from the group consisting of SEQ ID NOs: 1-16; and
   at least one peptide comprising an amino acid sequence selected from the group consisting of SEQ ID NOs: 17-54.
36. The vaccine composition of any of items 22-35, wherein the vaccine composition is administered in two doses, wherein the second dose is administered from at least two to four weeks after the first dose is administered.
37. The vaccine composition of item 36, wherein the initial dose comprises about 25 to about 500 micrograms (μg) peptides and wherein the second dose comprises the same amount of the peptides or double the amount of the peptides.
38. The composition of any of items 1-15 and 17-19, wherein the composition comprises one or more peptides comprising an amino acid sequence selected from the group consisting of (i)(a) SEQ ID NOs: 1, 3, 5, 10, 12, 14, 16, 17, 19, 23, 25, 28, 29, 32, 34, 35, 40, 44, 45, 47, 49, 51, and 53; and/or (i)(b) SEQ ID NOs: 2, 4, 6, 8, 9, 11, 13, 15, 18, 20, 21, 24, 26, 27, 30, 31, 33, 36, 37, 38, 39, 42, 46, 50 and 54 and/or (i)(c) SEQ ID NOs: 1, 3, 5, 8, 10, 12, 14, 15, 17, 19, 23, 25, 27, 29, 31, 34, 37, 42, 44, 46, 47, 48, 51, and 53.
39. The composition of any of items 1-15 and 17-19, wherein the composition comprises one or more peptides comprising an amino acid sequence selected from the group consisting of SEQ ID NOs: 1-16, and (i) 17-28, and/or (ii) 18-19, and/or (iii) 20-28, and/or (iv) 33-40 and/or (v) 41-44 and/or (vi) 53-54.
40. The composition of any of items 1-15 and 17-19, wherein the composition comprises one or more peptides comprising an amino acid sequence selected from the group consisting of SEQ ID NOs: 19-28 and/or 33-44.
40. The composition of any of items 1-15 and 17-19, wherein the composition comprises one or more peptides comprising an amino acid sequence selected from the group consisting of SEQ ID NOs: 10, 11, 12, 14, and 15.
41. The composition of any of items 1-15 and 17-19, wherein the composition comprises one or more peptides comprising an amino acid sequence selected from the group consisting of SEQ ID NOs: 1-16 and/or 23, 24 and 25.
42. The composition of any of items 1-15 and 17-19, wherein the vaccine composition comprises one or more peptides comprising an amino acid selected from the group consisting of (a) SEQ ID NOs: 10, 11, 12, 14, 15, 17, 23, 25, and/or 27; (b) SEQ ID NOs: 15 and 17-28; and/or (c) SEQ ID NOs: 17, 23, 25, and 27.
43. The composition of any of items 1-15 and 17-19, wherein the composition comprises:
   at least one peptide comprising an amino acid sequence selected from the group consisting of SEQ ID NOs: 1-16; and
   at least one peptide comprising an amino acid sequence selected from the group consisting of SEQ ID NOs: 17-54.
44. The method of any of claims 16-18 and 20-21, wherein the composition comprises one or more peptides comprising an amino acid sequence selected from the group consisting of (i)(a) SEQ ID NOs: 1, 3, 5, 10, 12, 14, 16, 17, 19, 23, 25, 28, 29, 32, 34, 35, 40, 44, 45, 47, 49, 51, and 53; and/or (i)(b) SEQ ID NOs: 2, 4, 6, 8, 9, 11, 13, 15, 18, 20, 21, 24, 26, 27, 30, 31, 33, 36, 37, 38, 39, 42, 46, 50 and 54 and/or (i)(c) SEQ ID NOs: 1, 3, 5, 8, 10, 12, 14, 15, 17, 19, 23, 25, 27, 29, 31, 34, 37, 42, 44, 46, 47, 48, 51, and 53.
45. The method of any of items 16-18 and 20-21, wherein the composition comprises one or more peptides comprising an amino acid sequence selected from the group consisting of SEQ ID NOs: 1-16, and (i) 17-28, and/or (ii) 18-19, and/or (iii) 20-28, and/or (iv) 33-40 and/or (v) 41-44 and/or (vi) 53-54.
46. The method of any of items 16-18 and 20-21, wherein the composition comprises one or more peptides comprising an amino acid sequence selected from the group consisting of SEQ ID NOs: 19-28 and/or 33-44.
47. The method of any of items 16-18 and 20-21, wherein the composition comprises one or more peptides comprising an amino acid sequence selected from the group consisting of SEQ ID NOs: 10, 11, 12, 14, and 15.
48. The method of any of items 16-18 and 20-21, wherein the composition comprises one or more peptides comprising an amino acid sequence selected from the group consisting of SEQ ID NOs: 1-16 and/or 23, 24 and 25.
49. The method of any of items 16-18 and 20-21, wherein the vaccine composition comprises one or more peptides comprising an amino acid selected from the group consisting of (a) SEQ ID NOs: 10, 11, 12, 14, 15, 17, 23, 25, and/or 27; (b) SEQ ID NOs: 15 and 17-28; and/or (c) SEQ ID NOs: 17, 23, 25, and 27.
50. The method of any of items 16-18 and 20-21, wherein the composition comprises: at least one peptide comprising an amino acid sequence selected from the group consisting of SEQ ID NOs: 1-16; and at least one peptide comprising an amino acid sequence selected from the group consisting of SEQ ID NOs: 17-54.
51. The method of any of items 16, 20, or 21, wherein the composition is administered in two doses, wherein the second dose is administered from at least two to four weeks after the first dose is administered.
52. The method of any of items 16, 20, or 21, wherein the initial dose comprises about 25 to about 500 micrograms (μg) peptides and wherein the second dose comprises the same amount of the peptides or double the amount of the peptides.

### Sequence Listing

Peptides were prepared by standard solid-state synthesis commercial methods. Each peptide was characterized as to amino acid content and purity. Peptides with the amino acid sequences listed below were thus synthesized. In these sequences, as in others of the present description, an amino acid beginning in a capital letter is an L-isomer amino acid, while one in a lower-case letter is a D-isomer.

All sequences are amino acid sequences, and all are artificial.

In the below table, please note:
nF/F*=para-nitro-L-Phenylalanine (bolded to make it easier to identify)
nL=L-Norleucine (bolded to make it easier to identify)

Amino acids in capital letters are L-isomers, and amino acid(s) in lower case are D-isomer amino acids.

| SEQ ID | |
|---|---|
| NO. | Sequence |
| 1 | AINGITNKVNSVIE-SSppppC |
| 2 | CppppSS-AINGITNKVNSVIE |
| 3 | RGFFGAIGFIEGGW-TSppppC |
| 4 | CppppST-RGFFGAIGFIEGGW |
| 5 | CppppST-NIHPITIGKSPKYVKS |
| 6 | NIHPITIGKSPKYVKS-TSppppC |
| 7 | CppppSS-nLATGLRNRP |
| 8 | nLATGLRNRP-SSppppC |
| 9 | CppppSS-RIENLNKKVDDG |
| 10 | RIENLNKKVDDG-SSppppC |
| 11 | CppppSS-DIWTYNAELLV |
| 12 | DIWTYNAELLV-SSppppC |
| 13 | CppppSS-EIGNGSFEFYHK |
| 14 | EIGNGSFEFYHK-SSppppC |
| 15 | CppppTS-IGYHANNST |
| 16 | IGYHANN-STppppC |
| 17 | nLSLLTEVET-TTppppC |
| 18 | CppppTT-nLSLLTEVET |
| 19 | ILRTQES-SSppppC |
| 20 | CppppSS-ILRTQES |
| 21 | WSSSASHDG-SSppppC |
| 22 | CppppSS-WSSSASHDG |
| 23 | CppppST-HIEESSSY |
| 24 | HIEESSSY-TSppppC |
| 25 | CppppST-SRDNWKGSNRP |
| 26 | SRDNWKGSNRP-STppppC |
| 27 | CppppST-TDGSASGQASTRILK |
| 28 | TDGSASGQASTRILK-TSppppC |
| 29 | CppppSS-DQVRESRNPGNAEIEDLI |
| 30 | DQVRESRNPGNAEIEDLI-STppppC |
| 31 | CppppSS-FLARSALILRGSVAHKS |
| 32 | FLARSALILRGSVAHKS-STppppC |
| 33 | CppppSS-HnLAIIKKF*TSGRQEKNPSL |
| 34 | HnLAIIKKF*TSGRQEKNPSL-STppppC |
| 35 | CppppST-RnLKWnLnLAnLKF*PITADK |
| 36 | RnLKWnLnLAnLKYPITADK-STppppC |
| 37 | CppppSS-VAYnLLERELVRKTRFLP |
| 38 | VAF*nLLERELVRKTRFLP-STppppC |
| 39 | CppppSS-VAGGTSSVF*IEVLHLTQG |
| 40 | VAGGTSSVF*IEVLHLTQG-TSppppC |
| 41 | CppppTS-YITRNQPEWFRNVLSIAP |
| 42 | F*ITRNQPEWFRNVLSIAP-STppppC |
| 43 | CppppSS-InLFSNKnLARLGKGYnLFESK |
| 44 | InLFSNKnLARLGKGYnLFESK-STppppC |
| 45 | CppppTT-DLEALnLEWLKTRPILSPLTKG |
| 46 | DLEALnLEWLKTRPILSPLTKG-TTppppC |
| 47 | CppppTS-ILGFVFTLTVPSERGLQRRRF* |
| 48 | ILGFVFTLTVPSERGLQRRRF-TSppppC |
| 49 | CppppTT-LQAYQKRnLGVQnLR |
| 50 | LQAYQKRnLGVQnLR-TTppppC |
| 51 | CppppTT-IIGILHLILWILDRLFFKSIF*RLF |
| 52 | IIGILHLILWILDRLF*FKSIYRLF-TTppppC |
| 53 | CppppTT-AADLKSTQEAING |
| 54 | AADLKSTQEAING-TTppppC |
| 55 | Cpppp |
| 56 | ppppC |

1. A polypeptide immunogenic composition comprising:
(A) at least one mimetic peptide comprising: (i) at least one consecutive amino acid sequence of conserved type A & B influenza viruses or a processed subspecies thereof, selected from the HA1 and/or HA2 protein domain groups and/or Neuraminidase (n1-N10) protein domain and/or Nucleocapsid protein domains and/or the M1 and/or M2 pore protein domain and/or the basic Polymerases B1 and/or B2 protein domains of the influenza viruses synthesized covalently to (ii) a 7 amino-acid spacer moiety, and conjugated to (iii) an immunogenic carrier coupled to said mimetic peptide(s),
wherein the mimetic peptide(s) comprises a chemically synthesized and/or modified amino acid and/or amino acid epitope that augments the immunogenicity of the polypeptide immunogen, and
wherein the amino acid sequence is from about 6 amino acid residues to about 30 amino acids, comprising a mixture of L- and D-enantiomeric amino acids.

2. The polypeptide immunogenic composition according to claim 1, further comprising
at least 3 to 5 oligopeptides comprising amino acid sequences and/or mimetic sequences selected from the group consisting of SEQ ID NOs: 1-16 and amino acid sequences and/or mimetic sequences selected from the group consisting of SEQ ID NOs: 17-54; and
a pharmaceutically acceptable carrier.

3. The polypeptide immunogenic composition according to claim 1, wherein the at least one mimetic peptide comprises:
at least one oligopeptide having an overall length of from 6-50 amino acids, optionally from 6-30 amino acids, selected from the group consisting of SEQ ID NOs: 1-16 and amino acid sequences and/or mimetic sequences selected from the groups consisting of SEQ ID NOs: 17-54 and a pharmaceutically acceptable carrier in a nano-sized emulsified preparation suitable for parenteral or mucosal application(s).

4. The polypeptide immunogenic composition according to claim 1, wherein at least the mimetic peptide(s) administered include a mixture of peptides selected from the group consisting of SEQ ID NO:1 to SEQ ID: NO:54.

5. The polypeptide immunogenic composition according to claim 1, wherein the immunogenic carrier is selected from the group consisting of toxoidal proteins like diphtheria toxoid (DT), tetanus toxoid (TT), pertussin toxoid (PT), or recombinant toxoidal-like protein, a non-toxic but immunogenic recombinant Tetanus (non-toxoidal) protein, and particulate carriers such as the nanoparticulate calcium phosphate (nCAP) carrier substance, with or without ancillary protein carriers.

6. (canceled)

7. An immunogenic composition comprising a therapeutically effective amount of the polypeptide immunogenic composition according to claim 1, and a pharmaceutically acceptable carrier.

8. The immunogenic composition according to claim 7, wherein the pharmaceutically acceptable carrier comprises an emulsion of an aqueous phase, in which the at least one mimetic peptide is selected from the group consisting of SEQ ID NOs: 1-54 is dissolved or in suspension, and
an oily phase,
wherein such aqueous emulsion comprises from 25% to 50% by weight of the composition.

9. The immunogenic composition according to claim 7, wherein said oily phase comprises at least one or more of squalene, squalane, sorbitan monooleate, Polysorbate 40, Poly Sorbate 80, and polysorbates.

10. The immunogenic composition according to claim 7, wherein said oily phase comprises at least one emulsifier agent capable of enabling oil/water (o/w) or water/oil (w/o) mixtures to form nano-sized emulsions (<500 nanometers); stable at 2-6° C. for at least one year or more.

11. The immunogenic composition according to claim 7, wherein either said oily phase or said aqueous phase contains at least one adjuvant.

12. The immunogenic composition according to claim 11, wherein said adjuvant is selected from the group consisting of: cyclic-di-purine mononucleotides, cyclic diguanylate, Imiquimod, cyclic diadenylate, Isoprinosine, trehalose dimycolate, QS-21, alpha-galactosylceramide (C-GalCer), Polyinosinic-polycytidylic acid (poly I:C), non-methylated CpG oligodeoxynucleotides (ODN), alpha-glucosylceramide (C-GluCer), and combinations thereof.

13-28. (canceled)

29. The vaccine composition of claim 22, wherein the vaccine composition comprises one or more peptides comprising an amino acid sequence selected from the group consisting of (i)(a) SEQ ID NOs: 1, 3, 5, 10, 12, 14, 16, 17, 19, 23, 25, 28, 29, 32, 34, 35, 40, 44, 45, 47, 49, 51, and 53; and/or (i)(b) SEQ ID NOs: 2, 4, 6, 8, 9, 11, 13, 15, 18, 20, 21, 24, 26, 27, 30, 31, 33, 36, 37, 38, 39, 42, 46, 50 and 54 and/or (i)(c) SEQ ID NOs: 1, 3, 5, 8, 10, 12, 14, 15, 17, 19, 23, 25, 27, 29, 31, 34, 37, 42, 44, 46, 47, 48, 51, and 53.

30. The vaccine composition of claim 22, wherein the vaccine composition comprises one or more peptides comprising an amino acid sequence selected from the group consisting of SEQ ID NOs: 1-16, and (i) 17-28, and/or (ii) 18-19, and/or (iii) 20-28, and/or (iv) 33-40, and/or (v) 41-44, and/or (vi) 53-54.

31-37. (canceled)

38. The immunogenic composition of claim 7, wherein the composition comprises one or more peptides comprising an amino acid sequence selected from the group consisting of (i)(a) SEQ ID NOs: 1, 3, 5, 10, 12, 14, 16, 17, 19, 23, 25, 28, 29, 32, 34, 35, 40, 44, 45, 47, 49, 51, and 53; and/or (i)(b) SEQ ID NOs: 2, 4, 6, 8, 9, 11, 13, 15, 18, 20, 21, 24, 26, 27, 30, 31, 33, 36, 37, 38, 39, 42, 46, 50 and 54 and/or (i)(c) SEQ ID NOs: 1, 3, 5, 8, 10, 12, 14, 15, 17, 19, 23, 25, 27, 29, 31, 34, 37, 42, 44, 46, 47, 48, 51, and 53.

39. The immunogenic composition of claim 7, wherein the immunogenic composition comprises one or more peptides comprising an amino acid sequence selected from the group consisting of SEQ ID NOs: 1-16, and (i) 17-28, and/or (ii) 18-19, and/or (iii) 20-28, and/or (iv) 33-40, and/or (v) 41-44, and/or (vi) 53-54.

40. The immunogenic composition of claim 7, wherein the immunogenic composition comprises one or more peptides comprising an amino acid sequence selected from the group consisting of SEQ ID NOs: 19-28 and/or 33-44.

41. The immunogenic composition of claim 7, wherein the immunogenic composition comprises one or more peptides comprising an amino acid sequence selected from the group consisting of SEQ ID NOs: 10, 11, 12, 14, and 15.

42. The immunogenic composition of claim 7, wherein the immunogenic composition comprises one or more peptides comprising an amino acid sequence selected from the group consisting of SEQ ID NOs: 1-16 and/or 23, 24 and 25.

43. The immunogenic composition of claim 7, wherein the immunogenic composition comprises one or more peptides comprising an amino acid selected from the group consisting of (a) SEQ ID NOs: 10, 11, 12, 14, 15, 17, 23, 25, and/or 27; (b) SEQ ID NOs: 15 and 17-28; and/or (c) SEQ ID NOs: 17, 23, 25, and 27.

44. The immunogenic composition of claim 7, wherein the immunogenic composition comprises:
at least one peptide comprising an amino acid sequence selected from the group consisting of SEQ ID NOs: 1-16; and
at least one peptide comprising an amino acid sequence selected from the group consisting of SEQ ID NOs: 17-54.

45-53. (canceled)
